(19) United States
(12) Patent Application Publication
(10) Pub. No.: US 2025/0242149 A1
(43) Pub. Date: Jul. 31, 2025

(54) STEERABLE CATHETER ASSEMBLIES

(71) Applicant: ABIOMED, Inc., Danvers, MA (US)

(72) Inventors: Alexander Ship, Danvers, MA (US); Christopher W. Sheils, Danvers, MA (US); Qingchao Kong, Danvers, MA (US); Christopher Theodore Bazdanes, Danvers, MA (US); Jeremy Yigal Nezaria, Danvers, MA (US); Iain Zwiebel, Danvers, MA (US); Jennifer Selingo, Danvers, MA (US); Lindsay Baxter, Danvers, MA (US)

(73) Assignee: ABIOMED, Inc., Danvers, MA (US)

(21) Appl. No.: 19/037,101

(22) Filed: Jan. 25, 2025

Related U.S. Application Data
(60) Provisional application No. 63/742,671, filed on Jan. 7, 2025.
(60) Provisional application No. 63/726,997, filed on Dec. 2, 2024.
(60) Provisional application No. 63/625,779, filed on Jan. 26, 2024.

(51) Int. Cl. A61M 60/865; A61M 60/13; A61M 60/414; A61M 60/508

(52) U.S. Cl. CPC A61M 60/865; A61M 60/13; A61M 60/414; A61M 60/508; A61M 2205/0233; A61M 2205/04; A61M 2205/103; A61M 2205/50; A61M 2210/125

(57) ABSTRACT

Steerable catheter assemblies, such as, steerable intracardiac blood pump assemblies are provided.

### CROSS REFERENCE TO RELATED APPLICATIONS

This application claims the priority of and benefit from U.S. Provisional Application No. 63/625,779, filed Jan. 26, 2024, U.S. Provisional Application No. 63/726,997, filed Dec. 2, 2024, and U.S. Provisional Application No. 63/742,671, filed Jan. 7, 2025, each of which is incorporated by reference herein.

### TECHNICAL FIELD

The present technology relates to steerable catheter assemblies, such as, steerable intracardiac blood pump assemblies.

### BACKGROUND

Catheter assemblies, such as, intracardiac blood pump assemblies can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

Catheter assemblies, such as, intracardiac blood pump assemblies can be introduced into the heart by a catheterization procedure. For example, with respect to an intracardiac blood pump assembly inserted into the left heart, an introducer sheath assembly may be inserted into the femoral artery through an arteriotomy to gain access to the artery and create an insertion path. A placement guidewire can be advanced into the artery along the insertion path. After the guidewire has been inserted into the artery, the pump assembly can be advanced over the guidewire and into the patient. Alternatively, the pump assembly can be inserted directly into the artery without a guidewire. The blood pump of the assembly can be inserted via a catheterization procedure through the femoral artery, into the ascending aorta, across the aortic valve and into the left ventricle. When deployed in the left heart, the pump assembly pulls blood from the left ventricle and expels blood into the ascending aorta.

### BRIEF SUMMARY

The present technology relates to steerable catheter assemblies, such as, steerable intracardiac blood pump assemblies.

In one aspect of the present technology an intracardiac blood pump system is provided. In this aspect, the intracardiac blood pump system comprises a blood pump, a catheter, a rotatable shaft, and a steering mechanism. The blood pump comprises a proximal portion and a distal portion. The catheter is coupled to the proximal portion of the blood pump and the catheter has a proximal end and a distal end and an inner lumen extending from the proximal end to the distal end. The rotatable shaft includes a proximal end and a distal end, wherein the shaft extends through the inner lumen of the catheter and the distal end of the shaft is coupled to the proximal portion of the blood pump. The steering mechanism is coupled to a portion of the shaft, wherein the steering mechanism is configured to rotate the shaft to rotate the blood pump.

In some aspects of the system, the steering mechanism is proximal of the catheter.

In some aspects of the system, the steering mechanism comprises a motor configured to rotate the shaft.

In some aspects of the system, the steering mechanism comprises: a torque input mechanism; and a torque transfer mechanism configured to transfer torque inputted to the torque input mechanism to the shaft to rotate the shaft.

In some aspects of the system, the torque input mechanism comprises a component that is manually rotatable by a user.

In some aspects of the system, the steering mechanism comprises: a housing; a first gear rotatably mounted in the housing and configured to be attached to the portion of shaft; a gear shaft at least partially disposed in the housing; and a second gear attached to the gear shaft and disposed within the housing such that the second gear engages the first gear, wherein when the gear shaft is rotated, the second gear rotates the first gear such that the shaft is rotated.

In some aspects of the system, the shaft comprises a torque coil.

In some aspects of the system, the shaft comprises a lumen extending from the proximal end to the distal end of the shaft.

In some aspects of the system, the system further comprises at least one of: one or more conductors extending through the lumen of the shaft and electrically coupled to one or more components of the blood pump; and/or an optical fiber extending through the lumen of the shaft and coupled to an optical sensor of the blood pump.

In some aspects of the system, the system further comprises a coupling mechanism configured to rotatably couple the proximal portion of the blood pump to the distal end of the catheter.

In some aspects of the system, the coupling mechanism is a slip ring.

In some aspects of the system, the slip ring electrically couples a first electrically conducting wire to a second electrically conducting wire, the first electrically conducting wire disposed in the inner lumen of the catheter and extending proximally from the slip ring toward the proximal end of the catheter and the second electrically conducting wire extending distally from the slip ring toward the blood pump.

In some aspects of the system, the system further comprises a controller, wherein the first electrically conducting wire is electrically coupled to the controller.

In some aspects of the system, the blood pump further comprises a motor and the second electrically conducting wire is electrically coupled to the motor.

In some aspects of the system, the blood pump further comprises a sensor and the second electrically conducting wire is electrically coupled to the sensor.

In some aspects of the system, the blood pump comprises a bend between the proximal portion and the distal portion.

In another aspect of the present technology, an intracardiac blood pump system is provided. In this aspect, the intracardiac blood pump system comprises a blood pump, a catheter, and a coupling mechanism. The blood pump comprises a proximal portion and a distal portion. The catheter is coupled to the proximal portion of the blood pump, and the catheter has a proximal end and a distal end and an inner lumen extending from the proximal end to the distal end. The coupling mechanism is configured to rotationally couple the proximal portion of the blood pump to the distal end of the catheter.

In some aspects of the system, the coupling mechanism comprises a first portion and a second portion, wherein the first portion is configured to be rotatable relative to the second portion, and wherein the first portion is coupled to the proximal portion of the blood pump and the second portion is coupled to the distal end of the catheter.

In some aspects of the system, the coupling mechanism comprises a bore extending through the coupling mechanism from a proximal end to a distal end of the coupling mechanism.

In some aspects of the system, the system further comprises a rotatable shaft including a proximal end and a distal end, wherein the shaft extends through the inner lumen of the catheter and the distal end of the shaft is coupled to the proximal portion of the blood pump.

In some aspects of the system, the shaft extends through the bore.

In some aspects of the system, the coupling mechanism is a slip ring.

In some aspects of the system, the system further comprises: at least a first electrically conducting wire coupled to the first portion of the slip ring and extending distally from the first portion toward the blood pump; and at least a second electrically conducting wire coupled to the second portion of the slip ring and extending proximally from the second portion toward the proximal end of the catheter, wherein the slip ring is configured to electrically couple the first and second electrically conducting wires.

In some aspects of the system, the system further comprises: a controller, and wherein the blood pump further comprises a motor, and the first electrically conducting wire is coupled to the motor and the first portion of the slip ring, and the second electrically conducting wire is coupled to the second portion of the slip ring and the controller.

In some aspects of the system, the system further comprises: a controller, and wherein the blood pump further comprises a sensor and the first electrically conducting wire is coupled to the sensor and the second electrically conducting wire is coupled to the controller.

In another aspect of the present technology, a steering mechanism for steering a blood pump of an intracardiac blood pump system is provided. The steering mechanism comprises; a housing; a first gear rotatably mounted in the housing and configured to be attached to a portion of a rotatable shaft of the intracardiac blood pump system; a gear shaft at least partially disposed in the housing; and a second gear attached to the gear shaft and disposed within the housing such that the second gear engages the first gear, wherein when the gear shaft is rotated, the second gear rotates the first gear such that the rotatable shaft of the intracardiac blood pump system is rotated.

In some aspects of the steering mechanism, a first end of the gear shaft is coupled to a manually rotatable component configured to permit a user to rotate the gear shaft.

In some aspects of the steering mechanism, a first end of the gear shaft is coupled to a motor configured to rotate the gear shaft.

### BRIEF DESCRIPTION OF THE DRAWINGS

FIG. 1A illustrates an exemplary blood pump portion of an intracardiac blood pump assembly for left heart support in accordance with aspects of the present technology.

FIGS. 1B and 1C illustrate additional components of the exemplary intracardiac blood pump assembly of FIG. 1A in accordance with aspects of the present technology.

FIG. 1D illustrates a portion of the exemplary intracardiac blood pump assembly of FIG. 1A including a securement device in accordance with aspects of the present technology.

FIG. 1E illustrates the blood pump portion of the exemplary intracardiac blood pump assembly of FIG. 1A inserted into the patient in accordance with aspects of the present technology.

FIG. 2A illustrates a steerable blood pump assembly in accordance with aspects of the present technology.

FIGS. 2B-2E illustrate alterative implementations of the steerable blood pump assembly of FIG. 2A in accordance with aspects of the present technology.

FIG. 3A illustrates a side cross-section of a portion of the steerable blood pump assembly of FIG. 2A including a coupling mechanism in accordance with aspects of the present technology.

FIG. 3B illustrates a side cross-section of a portion of the steerable blood pump assembly of FIG. 2A including a coupling mechanism comprising a slip ring in accordance with aspects of the present technology.

FIG. 3C illustrates a fiber optic rotary joint in accordance with aspects of the present technology.

FIGS. 4A-4D illustrate rotation and steering of a blood pump of the steerable blood pump assembly of FIG. 2A in accordance with aspects of the present technology.

FIGS. 5A-5D illustrate a steering mechanism for steering a blood pump assembly in accordance with aspects of the present technology.

FIG. 6 illustrates a steering mechanism including a lock in accordance with aspects of the present technology.

FIGS. 7A-7C illustrate a steering mechanism in accordance with aspects of the present technology.

FIGS. 8A-8C illustrate a steering mechanism in accordance with aspects of the present technology.

FIGS. 9A-9E illustrate a steering mechanism in accordance with aspects of the present technology.

FIGS. 10A and 10B illustrate alterative implementations of the steerable blood pump assembly of FIG. 2A in accordance with aspects of the present technology.

FIG. 11 illustrates a securement device configured to grip a portion of a catheter of a blood pump assembly in accordance with aspects of the present technology.

FIG. 12 illustrates a securement device configured to grip a portion of a catheter of a blood pump assembly in accordance with aspects of the present technology.

FIG. 13 illustrates a securement device with a grippable handle configured to grip a portion of a catheter of a blood pump assembly in accordance with aspects of the present technology.

FIG. 14 illustrates a securement device with a grippable handle configured to grip a portion of a catheter of a blood pump assembly in accordance with aspects of the present technology.

FIGS. 15A-15C illustrate a securement device with a grippable handle configured to grip a portion of a catheter of a blood pump assembly in accordance with aspects of the present technology.

FIGS. 16A-16B illustrate the securement device of FIGS. 15A-15C further including a coupling component coupled to the proximal end of the grippable handle of the securement device of FIGS. 15A-15C in accordance with aspects of the present technology.

FIG. 17 illustrates a gripping device for gripping a portion of a catheter of a blood pump assembly in accordance with aspects of the present technology.

FIG. 18 illustrates a gripping device for gripping a portion of a catheter of a blood pump assembly in accordance with aspects of the present technology.

FIG. 19 illustrates a gripping device for gripping a portion of a catheter of a blood pump assembly in accordance with aspects of the present technology.

FIG. 20 illustrates a portion of a blood pump assembly including a coupling mechanism in accordance with aspects of the present technology.

FIG. 21A illustrates a blood pump and a catheter for transferring torque in accordance with aspects of the present technology.

FIG. 21B is a cross-sectional view of the catheter of FIG. 21A along line A-A and illustrates a reinforcement structure of the catheter in accordance with aspects of the present technology.

FIG. 22 is a block diagram of a blood pump assembly in accordance with aspects of the present technology.

FIG. 23 is a flow chart of a method in accordance with aspects of the present technology.

### DETAILED DESCRIPTION

Aspects of the present technology are described in detail with reference to the drawing figures wherein like reference numerals identify similar or identical elements. It is to be understood that the disclosed aspects are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

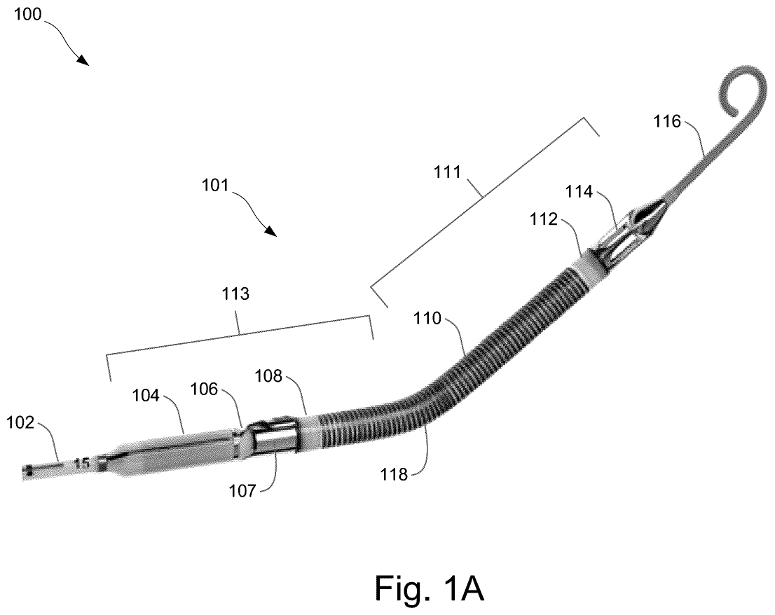

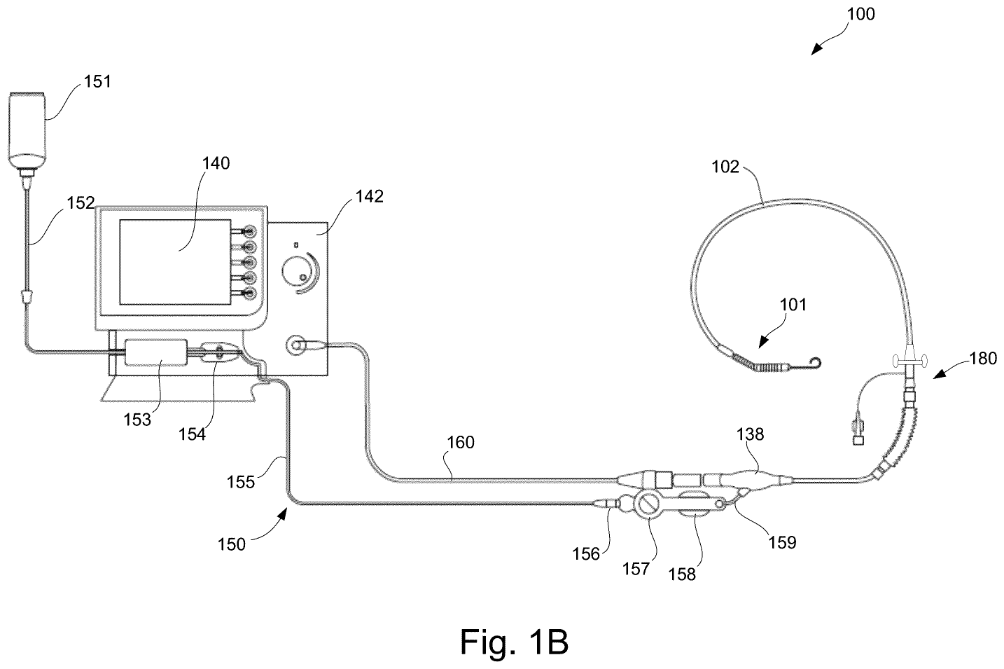

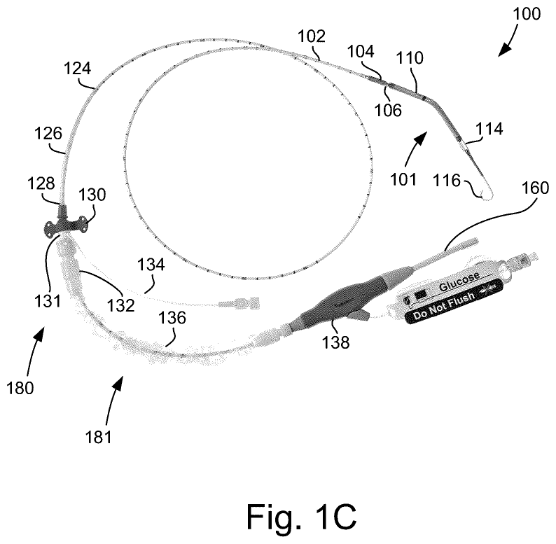

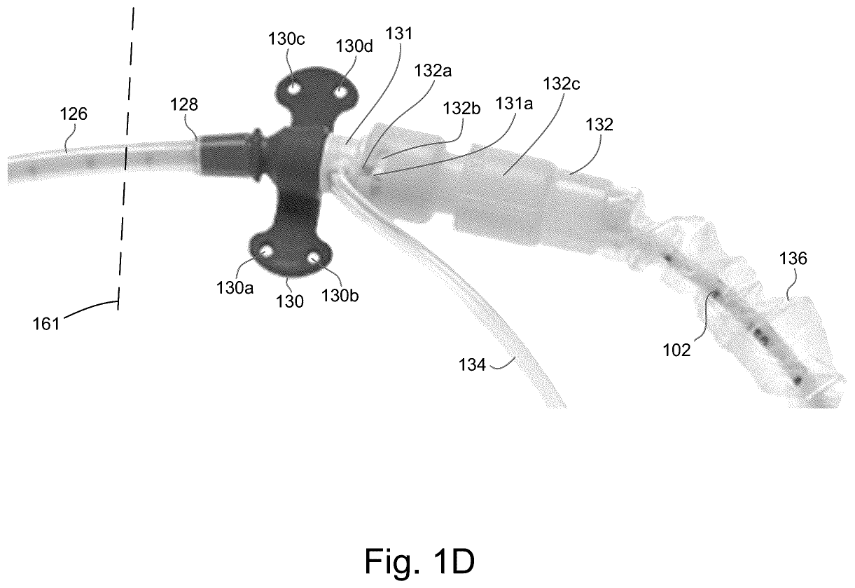

FIG. 1A depicts a blood pump portion 101 of an exemplary intracardiac blood pump assembly 100 adapted for left heart support, in accordance with aspects of the present technology. As shown in FIG. 1A, an intracardiac blood pump assembly 100 adapted for left heart support may include an elongate catheter 102, a motor housing 104, a cannula 110, a blood flow inlet 114 arranged at or near the distal end 112 of the cannula 110, a blood flow outlet 106 arranged at or near the proximal end 108 of the cannula 110, and an optional atraumatic extension 116 arranged at the distal end of the blood inflow cage 114. In one aspect, the inlet 114 is configured as a blood inflow cage and the outlet 106 is configured as a blood outflow cage. The motor housing 104, cannula 110, blood inflow cage 114, blood outflow cage 106 form a blood pump portion 101 of assembly 100. The blood pump portion 101 has a first portion 111 and a second portion 113. Portion 113 is proximal to portion 111. In one aspect, pre-formed bend 118 is disposed between the first portion 111 and the second portion 113.

In some aspects of the present technology, motor housing 104 houses a motor (not shown) that is configured to rotatably drive an impeller (not shown), thereby generating suction sufficient to draw blood into cannula 110 through the blood inflow cage 114, and to expel the blood out of cannula 110 through the blood outflow cage 106. In that regard, the impeller may be positioned distal of the blood outflow cage 106, for example, within the proximal end 108 of the cannula 110 or within a pump housing 107 coupled to the proximal end 108 of the cannula 110. In some aspects of the technology, rather than the impeller being driven by an onboard motor in motor housing 104, the impeller may instead be coupled to an elongate drive shaft (or drive cable) which is driven by a motor located external to the patient.

Catheter 102 may house electrical lines coupling the motor in motor housing 104 to one or more electrical controllers and/or sensors. Alternatively, where the impeller is driven by an external motor, an elongate drive shaft may pass through catheter 102. Catheter 102 may also include a purge fluid conduit, a lumen configured to receive a guidewire, one or more optical fibers (e.g., for sensing pressure), etc.

The blood inflow cage 114 may include one or more apertures or openings configured to allow blood to be drawn into cannula 110 when the motor in motor housing 104 is operating. Likewise, blood outflow cage 106 may include one or more apertures or openings configured to allow blood to flow from the cannula 110 out of the intracardiac blood pump assembly 100. Blood inflow cage 114 and outflow cage 106 may be composed of any suitable bio-compatible material(s). For example, blood inflow cage 114 and/or blood outflow cage 106 may be formed out of bio-compatible metals such as stainless steel, titanium, or biocompatible polymers such as polyurethane. In addition, the surfaces of blood inflow cage 114 and/or blood outflow cage 106 may be treated in various ways, including, but not limited to etching, texturing, or coating or plating with another material. For example, the surfaces of blood inflow cage 114 and/or blood outflow cage 106 may be laser textured.

Cannula 110 may include a flexible hose portion. For example, cannula 110 may be composed, at least in part, of a polyurethane material. In addition, cannula 110 may include a shape-memory material. For example, cannula 110 may comprise a combination of a polyurethane material and one or more strands or coils of a shape-memory material such as Nitinol. Cannula 110 may be formed such that it includes one or more bends or curves in its relaxed state, or it may be configured to be straight in its relaxed state. In that regard, as shown in the exemplary arrangement of FIG. 1A, the cannula 110 may have a single pre-formed anatomical bend 118 based on the portion of the left heart in which it is intended to operate. Despite this bend 118, the cannula 110 may nevertheless also be flexible, and may thus be capable of straightening (e.g., during insertion over a guidewire), or bending further (e.g., in a patient whose anatomy has tighter dimensions). Further in that regard, cannula 110 may include a shape-memory material configured to allow the cannula 110 to be a different shape (e.g., straight or mostly straight) at room temperatures, and to form bend 118 once the shape-memory material is exposed to the heat of a patient's body.

Atraumatic extension 116 may assist with stabilizing and positioning the intracardiac blood pump assembly 100 in the correct position in the patient's heart. Atraumatic extension 116 may be solid or tubular. If tubular, atraumatic extension 116 may be configured to allow a guidewire to be passed through it to further assist in the positioning of the intracardiac blood pump assembly 100. Atraumatic extension 116 may be any suitable size. For example, atraumatic extension 116 may have an outer diameter in the range of 4-8 Fr. Atraumatic extension 116 may be composed, at least in part, of a flexible material, and may be any suitable shape or configuration such as a straight configuration, a partially curved configuration, a pigtail-shaped configuration as shown in the example of FIG. 1, etc. Atraumatic extension 116 may also have sections with different stiffnesses. For example, atraumatic extension 116 may include a proximal section that is stiff enough to prevent it from buckling, thereby keeping the blood inflow cage 114 in the desired location, and a distal section that is softer and has a lower stiffness, thereby providing an atraumatic tip for contact with a wall of the patient's heart and to allow for guidewire loading. In such a case, the proximal and distal sections of the atraumatic extension 116 may be composed of different materials, or may be composed of the same material with the proximal and distal sections being treated to provide different stiffnesses.

Notwithstanding the foregoing, as mentioned above, atraumatic extension 116 is an optional structure. In that regard, the present technology may also be used with intracardiac blood pump assemblies and other intracardiac devices that include different types of extensions than those specifically described herein. These include extensions with different shapes than those described, extensions made of different materials than those described, extensions with different features than those described, etc. Likewise, the present technology may be used with intracardiac blood pump assemblies and other intracardiac devices that do not have a distal extension of any kind.

As shown in FIG. 1A, the distal end of catheter 102 is coupled the proximal end of proximal portion 113 (e.g., to motor housing 104). The proximal portion of catheter 102 is further coupled to additional components of blood pump assembly 100, as shown in FIG. 1B.

In this regard, as shown in FIG. 1B, in addition to blood pump 101 and catheter 102, the blood pump assembly 100 may further include a purging device or assembly 150, a controller 142 (e.g., an Automated Impella Controller® from Abiomed, Inc., Danvers, MA), a display 140, a connector cable 160, a plug or connector 138, and a repositioning unit 180. In some aspects, controller 142 includes display 140. Controller 142 comprises one or more processors. Controller 142 monitors and controls blood pump 101. During operation, purging device 150 delivers a purge fluid to blood pump 101 through catheter tube 102 to prevent blood from entering the motor (not shown) within motor housing 104. In some implementations, the purge fluid comprises a dextrose solution (e.g., 5% dextrose in water with 25 or 50 IU/mL of heparin). Connector cable 160 may provide electrical and/or optical connection(s) between blood pump 101 and controller 142. Plug 138 connects catheter tube 102, purging device 150, and connector cable 160. In some embodiments, plug 138 includes a memory for storing operating parameters in case the patient needs to be transferred to another controller 142. As will be described in greater detail below, repositioning unit 180 may be used as a tool to (position and) reposition blood pump 101 within a patient.

As shown, purging device 150 comprises a reservoir 151, purge fluid supply line 152, a purge cassette 153, a purge disc 154, purge tubing 155, a check valve 156, a pressure reservoir 157, an infusion filter 158, and a sidearm 159. Reservoir 151 may, for example, be a bag or a bottle. A purge fluid is stored in reservoir 151. A purge fluid spike at the end of purge fluid supply line 152 may be used to puncture reservoir 151 and connect the purge fluid in reservoir 151 to purge fluid supply line 152. Purge fluid supply line 152 carries the purge fluid from reservoir 151 to purge cassette 153. Purge tubing 155 carries the purge fluid from purge cassette 153 to blood pump 101.

Purge cassette 153 controls how the purge fluid in reservoir 151 is delivered to blood pump 101 and the flow path of the purge fluid from reservoir 151 to blood pump 101. For example, purge cassette 153 may include one or more valves (e.g., purge path diverters) for controlling a pressure and/or flow rate of the purge fluid. In addition to containing the components for delivering the purge fluid, purge cassette 153 also maintains the pressure barrier between the blood and the motor of blood pump 101 to prevent blood from entering the motor. Purge cassette 153 may contain a rack and pinion which is attached to a piston. Purge disc 154 includes one or more measuring device(s), such as pressure sensors (e.g., a pressure-sensing diaphragm) for measuring purge pressure of the purge fluid at blood pump 101. Controller 142 is connected to purge cassette 153 and purge disc 154. Purge disc 154 transmits pressure to controller 142 based on the purge pressure in purge tubing 155. A sensor in controller 142 measures the pressure so that it can be displayed on screen 140. Controller 142 may include a stepper motor. A tic (or step) represents stepper motor positions in units of microsteps, which are also called pulses. In some embodiments, a purge pressure/purge flow curve algorithm is deployed by the controller 142 in conjunction with the number of steps/minute of the stepper motor, and pressure measurements by purge disc 154 in order to calculate the corresponding purge flow rate.

As described above, purge tubing 155 provides a fluidic connection passing through purge cassette 153 to blood pump 101. In some embodiments, a Luer-lock connector is also provided, which facilitates a continuous fluid path or purge fluid path. A Luer-lock connector connects purge tubing 155 to a check valve 156 in the purge fluid path from the reservoir 151 to the blood pump 101. Pressure reservoir 157 provides additional filling volume during a purge fluid change. In some embodiments, pressure reservoir 157 includes a flexible rubber diaphragm that provides the additional filling volume by means of an expansion chamber. Infusion filter 158 helps prevent bacterial contamination and air from entering catheter tube 102. Sidearm 159 provides a fluidic connection between infusion filter 158 and plug 138.

During operation, controller 142 receives measurements from purge disc 154 and controls the stepper motor's number of ticks to control the purge pressure. In some embodiments, during operation, purge cassette 153 is placed in controller 142 and connected with the purge line to blood pump 101. As noted above, controller 142 controls and measures purge pressure and calculates purge flow rate via purge cassette 153 and/or purge disc 154. Controller 142 may also control the purge fluid supply. During operation, after exiting purging device 150 through sidearm 159, the purge fluid is channeled through purge lumens (not shown) within plug 138 and catheter tube 102. Sensor cables (not shown) within catheter tube 102, connector cable 160, and plug 138 provide an electrical connection between purge disc 154 and controller 142. Motor cables (not shown) within catheter tube 102, connector cable 160, and plug 138 provide an electrical connection between the motor within motor housing 104 and controller 142. During operation, controller 142 receives measurements from purge disc 154 through the sensor cables and controls the electrical power delivered to the motor within motor housing 104 through the motor cables. By controlling the power delivered to the motor within motor housing 104, controller 142 may control the speed of the motor within motor housing 104. In some embodiments, controller 142 includes safety features to prevent air from entering purge tubing 155. Controller 142 may include (or be in communication with) circuitry for monitoring the motor current for drops in current indicating air in the line. Controller 142 may include or be configured to generate warning sounds, lights or indicators to alert an operator of certain detected conditions, such as, but no limited to, pump position, suction events at the inlet, and disconnects or breaks in purge tubing 155 which may result in the introduction of air to the line.

In some aspects, assembly 100 may include one or more sensors or measurements devices configured to communicate with controller 142 to provide information associated with the operation of assembly 100 or a patient. In one aspect, assembly 100 may include an optical fiber (disposed through one or more of cable 160, plug 138, and catheter 102) that forms a pressure sensitive surface at its distal end. The pressure sensitive surface forms a pressure sensor which may be added to blood pump 101 near its inlet area 114. The pressure sensor is configured to measure a left ventricular blood pressure. Assembly 100 may implement multiple such pressure sensors (or other sensor types) at different locations (e.g., on or in pump 101, catheter 102, plug 138, etc.) throughout assembly 100 and controller 142 may be configured to perform different steps based on the information received for the sensors. In some aspects, additional sensor cables may be disposed within catheter tube 102, connector cable 160, and plug 138 to provide an electrical and/or optical connection between the one or more additional measuring devices and controller 142. As yet another example, one or more components of blood pump assembly 100 may be separated. For example, display 140 may be incorporated into another device in communication with controller 142 (e.g., wirelessly or through one or more electrical cables).

Display 140 is controllable by controller 142 to display useful information to the user of the blood pump assembly 100. For example, display 140 may be controlled to display many different types of information such as the characteristics of the blood pump assembly (e.g., blood pump type, serial number, software version, etc.), operation of the blood pump assembly (e.g., present blood pump speed (performance) setting, blood pump flow measurements, purging device measurements, a status indicator, sensor measurements, blood pump position detections and indications, etc.). Some of this information may be obtained from purge disc 154 or any of the other sensors described above or that may be used with assembly 100. Display 140 can also provide notifications to the user. For example, a notification may serve as an alert and include a statement describing the cause of the alert. In some embodiments, display 140 may be a touchscreen and a user may switch between screens by tapping button labels on display 140. In some embodiments, a user may use a separate input device, such as a mouse or a keyboard, to switch between screens.

As described above, assembly 100 may include a repositioning unit for repositioning the blood pump 101 within the patient. For example, FIG. 1C shows an exemplary implementation of blood pump assembly 100 with a repositioning unit or assembly 180, in accordance with aspects of the present technology.

In one aspect, the repositioning unit 180 may include repositioning sheath 126, fixation device or butterfly 130, hemostasis valve 131, securement device 132, and protective sleeve 136. In this aspect, between handle 138 and securement device 132, the catheter 102 is enclosed within a protective sleeve 136. Protective sleeve 136 may be configured to prevent contamination of catheter 102 as it is advanced in the distal direction for insertion into the patient's vasculature. Protective sleeve 136 may be comprised of any suitable material, and may be secured at its proximal and distal ends in any suitable manner. The distal end of protective sleeve 136 may be coupled to securement device 132. In the example of FIG. 1C, securement device 132 is coupled at its distal end to a hemostasis valve 131, which in turn is attached to butterfly 130. Hemostasis valve 131 may be integrated with butterfly 130, or may be removably coupled thereto. In addition, hemostasis valve 131 and securement device 132 may be incorporated into a single unit. Butterfly 130 is coupled at its distal end to the proximal end 128 of repositioning sheath 126. The distal end of repositioning sheath 126 is indicated with reference numeral 124. Repositioning sheath 126 is configured with a lumen sized to allow passage of at least catheter 102, but otherwise may have any suitable length and construction.

Generally, a blood pump, such as blood pump 101, may initially be inserted into the patient's vasculature via an introducer sheath assembly. Once the blood pump 101 has been inserted into the patient's vasculature, the operator may advance the blood pump 101 to its desired location in the body (e.g., left heart or right heart). As the catheter 102 advances further into the patient's vasculature, the distal end 124 of repositioning sheath 126 may be inserted into the introducer sheath assembly which acts as a conduit for repositioning sheath 126 to enter the patient's vasculature. In some aspects, the introducer sheath may be removed thereafter, for example, in the case of a tear away design, by tearing the introducer along its length. However, in other aspects, where introducer sheath assembly is an expandable design, it may remain in the body, either surrounding some or all of the repositioning sheath 126, or, in cases where the repositioning sheath 126 remains outside of the body, surrounding catheter 102.

Once repositioning sheath 126 has been fully inserted, the operator may secure it to the patient at or near the insertion site using butterfly 130. In that regard, butterfly 130 may be affixed to the patient (e.g., using adhesives or sutures) in order to secure the repositioning sheath 126 and securement device 132 relative to the patient. Thereafter, once the blood pump has been advanced to the desired location within the patient's body, the operator may use securement device 132 to restrict further movement of the catheter 102 and blood pump 101 within the patient. In that regard, securement device 132 may be any device suitable for optionally allowing and restricting movement of catheter 102 therethrough. In one aspect, the specific securement device 132 depicted in FIG. 1C is a Tuohy-Borst type device. However, other suitable securement devices may be used in place of securement device 132.

FIG. 1D shows an enlarged view of the blood pump assembly 100 and repositioning sheath assembly 180 of FIG. 1C. FIG. 1D reproduces a portion of blood pump assembly 100 and repositioning sheath assembly 180, oriented with its distal end to the left as though repositioning sheath 126 has been passed through a patient's skin surface (indicated by dashed line 161) from right to left. Oriented in this way, the proximal end 128 of repositioning sheath 126 would remain on the outside of the patient's body, and butterfly 130 may be secured to the patient's skin using sutures passed through suture eyelets 130a, 130b, 130c, and 130d.

The enlarged view of FIG. 1D shows a bayonet connection between hemostasis valve 131 and securement device 132. In that regard, the distal end of securement device 132 is configured such that it can be coupled to a proximal end of hemostasis valve 131 by pushing the two parts together and turning them relative to one another. In the example of FIG. 1D, the distal portion of securement device 132 has a cylindrical collar with one or more slots, and the proximal portion of hemostasis valve 131 has a cylindrical projection with one or more pegs 131a. When the cylindrical collar of securement device 132 is advanced over the cylindrical projection of hemostasis valve 131, each peg 131a will enter one of the slots in securement device 132 at an entrance point 132a. Then, by rotating the securement device 132 relative to hemostasis valve 131, each peg 131a will move toward an end point 132b of the slot, thus preventing securement device 132 from being pulled away from hemostasis valve 131 (without first rotating the two components in the opposite direction). In addition, the internal interface between the cylindrical collar of securement device 132 and hemostasis valve 131 may include a deformable seal or gasket (e.g., a rubber washer) to both provide a seal between the parts and to provide backpressure tending to prevent peg 131a from easily moving within the slot of securement device 132. Moreover, a detent may be provided at the end points 132b of each slot in securement device 132 so that peg 131a will tend to remain in the locked state.

As already noted, in the example of FIGS. 1C and 1D, the securement device 132 is a Tuohy-Borst device in which a barrel 132c is rotated to vary the amount of resistance imposed on whatever object (e.g., catheter 102) is within the securement device 132.

As described herein, the intracardiac blood pump assembly 100 may be inserted percutaneously. For example, when used for left heart support, intracardiac blood pump assembly 100 may be inserted via a catheterization procedure through the femoral artery or axillary artery, into the aorta, across the aortic valve, and into the left ventricle. An exemplary implementation of this positioning is shown in FIG. 1E. Once positioned in this way, the intracardiac blood pump assembly 100 may be controlled (e.g., using controller 142 to control the motor in housing 104 to rotate the impeller or rotor of blood pump 101) to deliver blood drawn in from the blood inflow cage 114, which sits inside the left ventricle, through cannula 110, out through the blood outflow cage 106, which sits inside the ascending aorta. In some aspects of the technology, intracardiac blood pump assembly 100 may be configured such that bend 118 will rest against a predetermined portion of the patient's vasculature when the intracardiac blood pump assembly 100 is in a desired location. Likewise, the atraumatic extension 116 may be configured such that it rests against a different predetermined portion of the patient's heart when the intracardiac blood pump assembly 100 is in the desired location.

While and/or after the blood pump 101 is inserted into the intended location (e.g., the left ventricle and ascending aorta in the case of a left heart pump) of the patient, the precise orientation and position of blood pump 101 may need to be adjusted to place the inlet 114 and outlet 106 in the desired positions relative to the body structures of the patient. For example, to avoid suction with the tissues and/or surfaces in the left ventricle, it may be desirable for the inlet 114 to be positioned in the free space of the left ventricle such that the inlet 114 does not contact the inner walls of the left ventricle or the chordae that actuate the mitral valve. This position may be achieved by orienting the distal portion 111 of the blood pump 101 to point toward the apex of the left ventricle and away from the chordae that actuate the mitral valve and having appropriate spacing away from the internal walls of the left ventricle.

In some instances, when the pump 101 is inserted into the patient's heart, the pump 101 may not be in the desired orientation to effectively pump blood and prevent suction events. This may occur for several reasons. For example, prior to being inserted into the patient, the catheter 102 and pump 101 may have an inherent or resting state shape. For purposes herein, the inherent or resting state shape is the shape the catheter 102 and pump 101 take when no external forces or stresses are being applied to the catheter 102 and pump 101. For example, when at rest and exterior to the patient, e.g., placed on a flat surface such as a table, the pump 101 and catheter 102 may be disposed in the same two-dimensional plane (e.g., in this case, defined by the surface of the table). Due to the inherent resting state shape of the catheter 102 and pump 101, when the pump 101 is inserted into the patient's heart, the pump 101 may not be in the desired orientation or position and thus may need to be adjusted to achieve the desired position. For example, the resting state shape of the catheter 102 and pump 101 may cause the distal portion 111 of the pump, including the inlet 114, to initially be biased toward and in contact with the ventricular walls or other structures in the left ventricle, which may cause the inlet 114 to be blocked or reduce its capacity to draw in blood into the cannula 110. As will be further appreciated, due to movement of the patient and/or movement of the pump during use, the position of the pump may need to be adjusted for continued use.

In some instances, to alter the position of the pump 101 within the patient, a user may attempt to grasp the portion of the catheter 102 that is exterior to the patient and apply torque to the catheter 102 to torsion/twist the catheter 102 and thereby rotate the cannula 110 to change the orientation of distal portion 111 of pump 101 within the patient's heart. Moreover, the user may pull (proximally) or push (distally) the catheter 102 into or out of the patient. Referring to FIG. 1C, the portion of the catheter 102 that is exterior to the patient when the pump 101 is inserted in the patient is indicated by reference numeral 181. Portion 181 is the portion of catheter 102 that is distal of plug 138 and proximal of fixation device or butterfly 130 (when butterfly 130 is affixed to the patient proximate to the access site. Some or all of portion 181 may be covered by protective sleeve 136 and/or a securement device, such as securement device 132.

In some instances, manually torquing and/or pulling/pushing portion 181 of catheter 102 in existing heart pump assemblies may be difficult for the user. For example, there may be a limited amount of torque that can safely be applied to the catheter 102 without kinking the catheter 102 or before the applied torque is released in an uncontrollable manner such that the catheter 102 recoils. Moreover, the exact torquing of the catheter 102 may not be known to the user while the user is torquing, and/or the adjusted position may not be permanent, which may cause the user to need to adjust further and/or stop to confirm the adjusted position. For example, after the user stops torquing the portion 181 of catheter 102, the resting state shape of the assembly 100 may cause the distal cannula portion 111 to return to an undesirable position where the inlet 114 is contacting the patient's body. Still further, portion 181 (being flexible and not restrained at all points, e.g., against the patient) may not be sufficiently stable to transfer torque appropriately through catheter 102 to cannula 110 or to permit the user to have sufficient precision in the amount of torque transferred to cannula 110 when portion 181 is torqued.

As described herein, the inventors have recognized the benefits of improving a user's ability to apply torque and adjust a position of the blood pump. For example, the present technology provides steerable heart pump assemblies that may permit a user to manipulate and steer the blood pump 101 to desired positions and orientations within the body of the patient. In some embodiments, the technology may allow the user to achieve an improved grip of a portion of the blood pump, to effectuate such torquing. In other embodiments, the described assemblies may allow the user to selectively apply torque to desired regions of the blood pump and/or the catheter. In still other embodiments, the technology may provide a feedback loop for users so that they can monitor and/or adjust the torque to achieve a desired position of the blood pump.

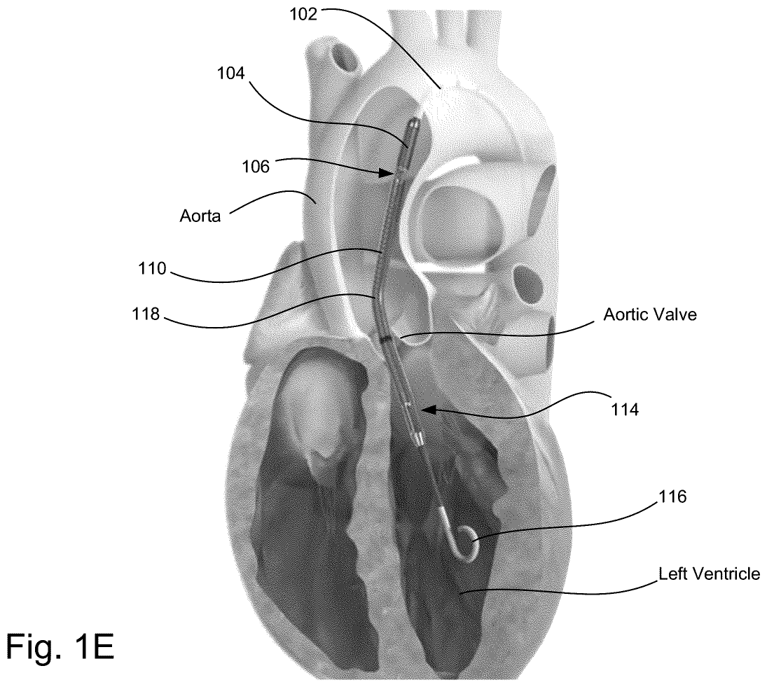

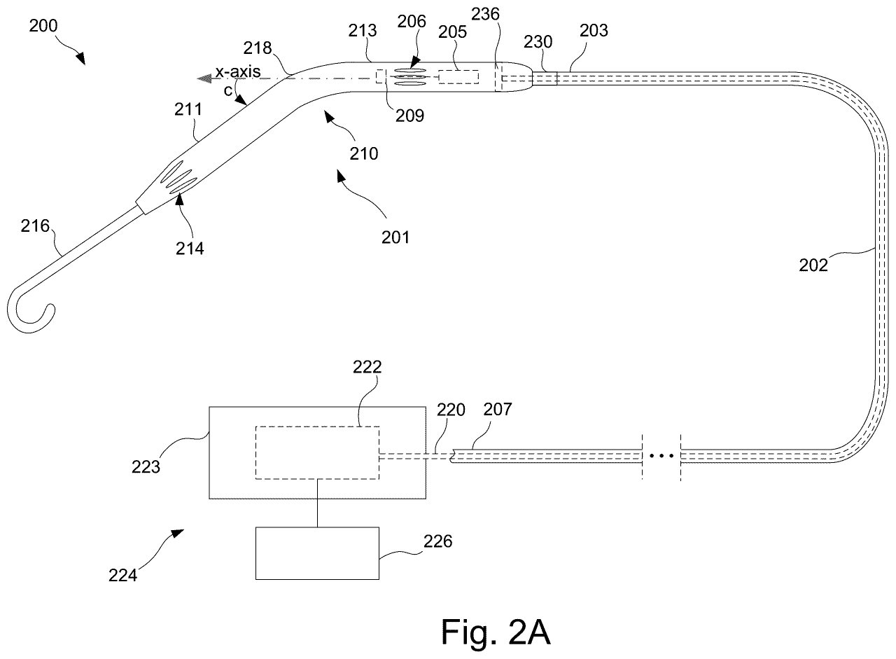

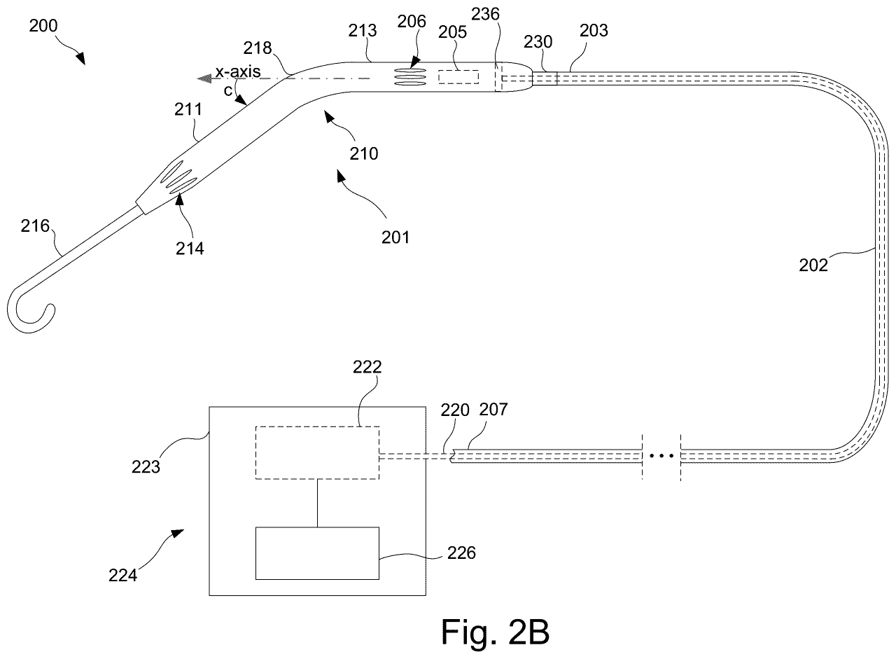

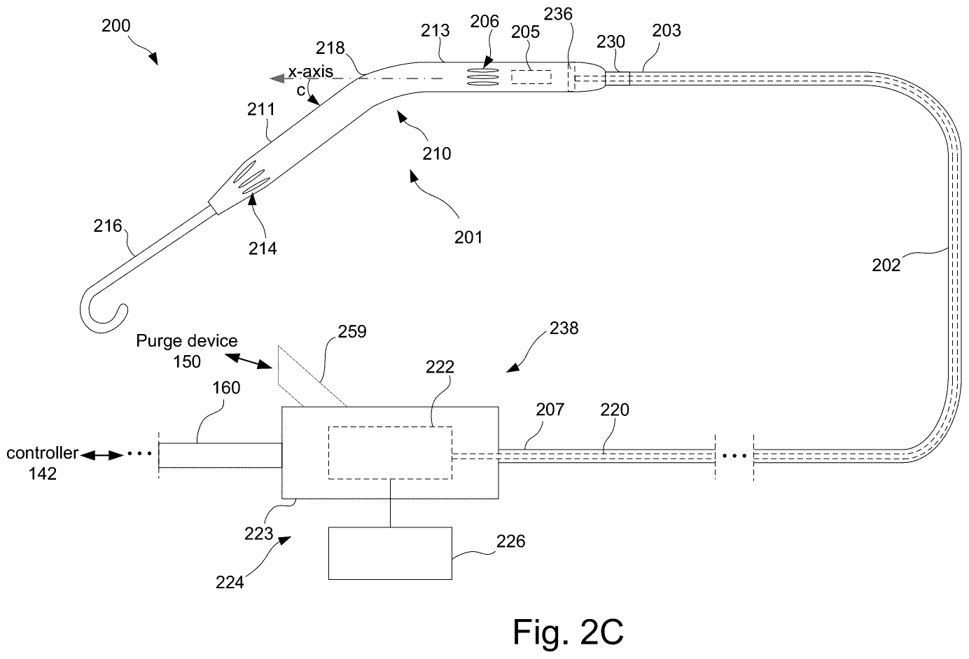

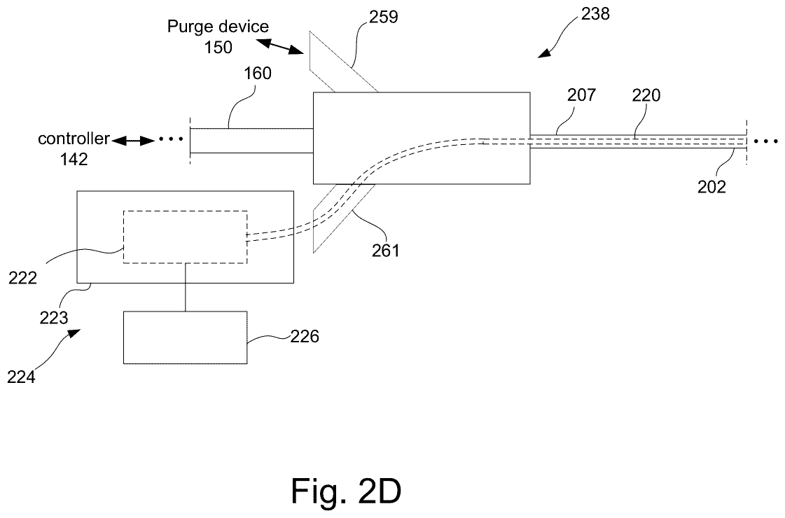

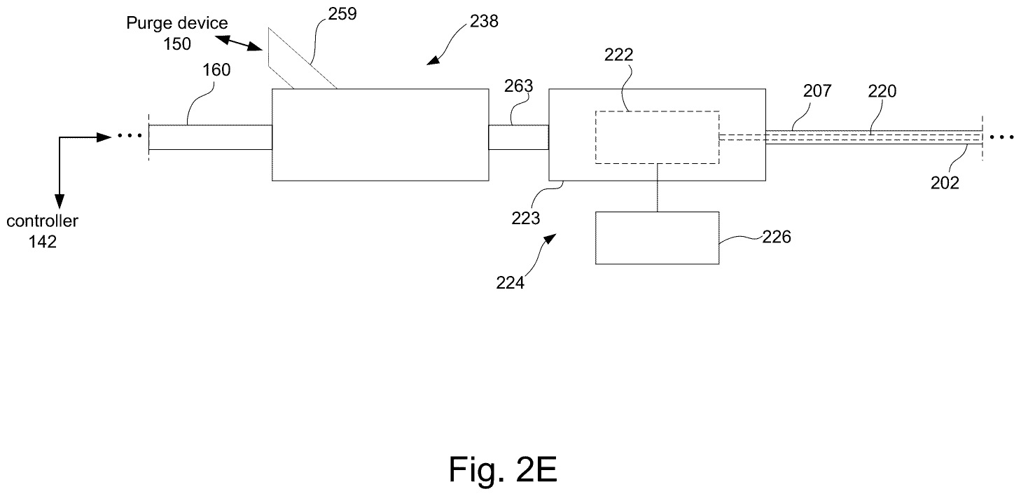

For example, referring to FIG. 2A, a steerable heart pump assembly 200 is shown in accordance with aspects of the present technology. In one aspect, assembly 200 may include a blood pump portion 201, a catheter 202, and a steering mechanism 224. Blood pump portion 201 may be coupled to a distal portion 203 of catheter 202. As described in greater detail herein, steering mechanism 224 may be disposed proximal of catheter 202 and coupled to a rotatable shaft 220 extending through catheter 202 and configured to rotate shaft 220 to steer blood pump 201. It is to be appreciated that, unless otherwise specified herein, blood pump 201 and catheter 202 may include any of the features of the blood pump portion 101 and catheter 102 of assembly 100 describe herein. Moreover, it is to be appreciated that, although not shown, assembly 200 may include other components, such as any of the component described above in relation to assembly 100. For example, assembly 200 may include repositioning unit 180, controller 142, handle 138, purge device 150, etc., of assembly 100.

Blood pump 201 may include a first (distal) portion 211 and a second (proximal) portion 213. Atraumatic tip 216 extends from the distal end of the distal portion 211. Blood pump 201 includes a cannula 210. Cannula 210 includes a pre-formed bend 218 between the distal portion 211 and the proximal portion 213, such that distal portion 211 extends from proximal portion 213 at a predetermined angle c. Angle c is measured relative to an x-axis that proximal portion 213 extends along. Blood pump 201 includes a motor 205 disposed near the proximal end of proximal portion 213. In one aspect, the motor 205 is disposed in a motor housing (not shown) similar to housing 104 that forms a portion of proximal portion 213. Motor 205 is configured to rotate a rotor or impeller 209 within the proximal portion 213 to cause blood to be pulled or drawn in to cannula 210 through inlet 214 and conveyed out of cannula 210 through outlet 206. Similar to cannula 110, in some aspects, cannula 210 may extend from an inflow cage (not shown) forming inlet 214 to a pump housing (not shown) disposed around rotor 209 or to an outflow cage (not shown) forming outlet 206. Proximal portion 213 of blood pump 201 is coupled to a distal portion 203 of catheter 202. Several electrically conducting wires (conductors) and/or optical fibers extend through the interior of catheter 202, for example, to carry data signals and power to/from motor 205 and one or more sensors (e.g., pressure sensors) of assembly 200. The wires may be in communication with a controller such as controller 142, described above.

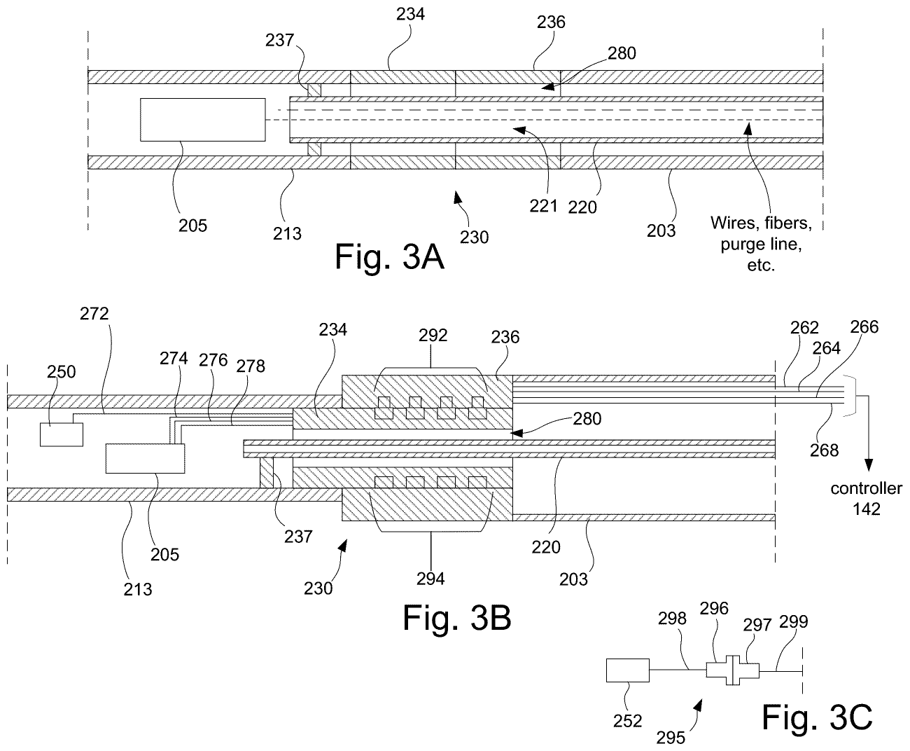

In one aspect, assembly 200 includes an elongate shaft 220 that is coupled to the steering mechanism 224 and extends through the interior lumen of catheter 202. The elongate shaft 220 is configured to be independently rotatable within catheter 202 and to transfer torque to blood pump 201, for example, to proximal portion 213. The transferred torque may be used to steer and rotate blood pump 201, as described in greater detail below. In some aspects, as shown in FIG. 3A, shaft 220 includes a hollow interior forming a lumen 221 configured to carry the wires and/or fibers that run through catheter 202. In one aspect, the shaft 220 is formed of one or more coils or springs. In one aspect, shaft 220 has a solid interior. In all aspects, shaft 220 is configured to transfer torque along its length and to be sufficiently flexible to bend with catheter 202 when assembly 200 is inserted through the vasculature of the patient. Shaft 220 may be made of a flexible metal, polymer, or composite material.

In some aspects, the shaft 220 may comprise a torque coil or cable including one or more layers of coils, such as multifilar coils. The torque coil may include a lumen 221 for the electrical wires and/or optical fibers of assembly 200 to extend through. The torque coil may have several properties and characteristics such that torque is selectively transferred by shaft 220. For example, torque coil may comprise one or more layers selected to permit the torque coil to transfer torque when rotated in only one direction (clockwise or counterclockwise) or in multiple direction (clockwise and counterclockwise). The number of coil layers, coil pitch, filars, and rotation directions can each be selected according the specific torque transfer characteristics required for satisfactory operation of shaft 220.

In one aspect, one or more of the torque coil properties may be selected such that the torque coil can have an asymmetric torque response when comparing the torque inputted at the proximal end of the torque coil at steering mechanism 224 to the corresponding torque outputted at the distal end of the torque coil. For example, the torque coil may be configured such that if a first amount of torque is inputted at the proximal end of the torque coil, the torque coil outputs a second amount of torque at the distal end of the torque coil, where the second amount of torque is greater than the first amount of torque. In this way, a small amount of torque may be inputted to the torque coil at the user end and a large amount of torque may be outputted by the coil at the distal end to steer the cannula 210.

As described herein, assembly 200 may include a steering mechanism 224 for rotating shaft 220 to permit a user to steer blood pump 201 to a desired position.

Referring to FIG. 2A, in some aspects of the present technology, steering mechanism 224 may include a torque input mechanism 226 (e.g., manually controllable by the user or by controller 142) and a torque transfer mechanism 222. Torque transfer mechanism 222 is configured to transfer torque, e.g., inputted from mechanism 226, to shaft 220 to rotate shaft 220 (clockwise or counterclockwise) relative to catheter 202 and thereby steer blood pump 201. Torque transfer mechanism 222 and torque input mechanism 226 are described in greater detail below. As shown, in one aspect, torque transfer mechanism 222 may be disposed within a housing 223 and torque input mechanism 226 may be disposed exterior to housing 223 (e.g., on or near an exterior surface of housing 223) and coupled to torque transfer mechanism 222.

Alternatively, as shown in FIG. 2B, in another aspect, torque input mechanism 226 may be disposed within housing 223 with torque transfer mechanism 222.

It is to be appreciated that although torque input mechanism 226 and torque transfer mechanism 222 are illustrated and described as separate mechanisms, in some aspects, mechanisms 222 and 226 may be combined.

In one aspect, mechanism 226 may be a motor (e.g., an electrical motor) that is controllable to rotate shaft 220. The motor may be controlled by the controller (e.g., controller 142) of assembly 200 via user input to the controller or by another controller integrated in or proximate to housing 223.

In another aspect, mechanism 222 may be a motor (e.g., an electrical motor) that is controllable (e.g., by controller 142 or another controller) to rotate shaft 220. In this aspect, steering mechanism 224 may not include mechanism 226. The motor may be disposed in the housing 223.

In another aspect, mechanism 226 may be a mechanism for receiving manually applied torque (or another force converted into torque) directly from a user. For example, mechanism 226 may include a knob, gear, thumb-wheel, lever, screw, slide, etc. that is configured to be manipulated or actuated (e.g., rotated) by the user to input to torque (or another force converted to torque) to torque transfer mechanism 222.

The steering mechanism 224 may be implemented in several different ways in blood pump assembly 200. For example, in one aspect, steering mechanism 224 is implemented in (or as) a plug of the assembly, such as plug 138 described above. This aspect is shown in FIG. 2C. In this aspect, assembly 200 includes a plug 238 having some or all of the features of plug 138 described herein. In this regard, plug 238 is coupled to cable 160 and catheter 202 and includes a sidearm 259 (e.g., a tube or port) for coupling to the purge device 150. In this aspect, the housing 223 forms the housing of plug 238 and torque transfer mechanism 222 is internal to the housing 223 of the plug 238. Moreover, the torque input mechanism 226 may be disposed at least partially exterior to housing 238 and extend from the exterior of the housing 238 into the housing 238. In this aspect, the wires, fibers, purge line, and any other assembly elements that enter the plug 238 from cable 160 and purge line 155, may conveniently further enter into the lumen of catheter 202 either through the lumen of shaft 220 or exterior to shaft 220 within the lumen of catheter 202.

In another aspect, steering mechanism 224 may be implemented as a separate structure than plug 238. For instance, an example of this aspect is shown in FIG. 2D. In the example of FIG. 2D, the plug 238 may include an additional sidearm 261 (e.g., a tube or port). The shaft 220 is coupled to steering mechanism 224 (e.g., to mechanism 222) and exits housing 223 (e.g., via an aperture in the housing) of steering mechanism 224 and enters through sidearm 261 into plug 238. Plug 238 is coupled to proximal portion 207 of catheter 202 and the shaft 220 extends through the interior of plug 238 and enters catheter 202. In this example, the wires, fibers, and purge line in plug 238 enter catheter 202 (via cable 160 and sidearm 259) and are exterior to shaft 220.

Another example where steering mechanism 224 is implemented as a separate structure from plug 238 is shown in FIG. 2E. In the example of FIG. 2E, the steering mechanism 224 is distal of plug 238 and coupled to plug 238 by a coupling element 263 (e.g., a conduit, tube, cable, etc.). In this example, the wires, fibers, and purge line in plug 238 enter the housing 223 of steering mechanism 224 via coupling element 263 and from there enter the lumen of shaft 220 (or the lumen of catheter 202 but exterior to shaft 220). The proximal portion 207 of catheter 202 is coupled to a distal end of the housing 223 of steering mechanism 224.

In any of the aspects and examples described herein, the assembly 200 may include a coupling mechanism that is configured to permit the proximal portion 213 of blood pump 201 to rotate relative to the distal portion 203 of catheter 202 without (or reducing) applying or storing torque in catheter 202. For example, a coupling mechanism 230 is shown in FIG. 2A. Coupling mechanism 230 is configured rotatably connect the proximal portion 213 of blood pump 201 to the distal portion 203 of catheter 202. In one aspect, coupling mechanism 230 comprises a bearing assembly or a bushing assembly. In some aspects, as described in greater detail herein, the coupling mechanism 230 may comprise a rotary joint, such as a slip ring. The slip ring may include a through hole or bore through its center.

One aspect of coupling mechanism 230 as implemented in assembly 200 is shown in FIG. 3A. As shown, in this aspect, coupling mechanism 230 includes a first (distal) portion 234 and a second (proximal) portion 236. Portion 234 of coupling mechanism 230 is fixedly coupled to (e.g., the proximal end of) proximal portion 213 of blood pump portion 201. Portion 236 of coupling mechanism 230 is fixedly coupled to the distal portion 203 (e.g., the distal end) of catheter 202. Portion 234 and portion 236 of coupling mechanism 230 are configured to be rotatable relative to each other. In one aspect portions 234, 236 are rotatably connected by one or more bearings. Thus, coupling mechanism 230 is configured to permit the proximal portion 213 of blood pump 201 to rotate relative to the distal portion 203 of catheter 202. The shaft 220 and electrical wires/optical fibers within the catheter 202 (e.g., disposed within shaft 220) extend through a bore or through hole 280 within the coupling mechanism 230 into the interior of blood pump 201. A distal portion of shaft 220 is fixedly coupled to the proximal portion 213 of blood pump 201 to transfer torque applied to shaft 220 to proximal portion 213 of blood pump 201. In one aspect, the distal portion of shaft 220 is fixedly coupled to the interior of proximal portion 213 via one or more mounts or yokes 237.

As described above, coupling mechanism 230 may comprise a through-hole slip ring configured to electrically and rotationally couple one or more electrically conducting wires proximal of coupling mechanisms 230 to one or more electrically conducting wires distal of coupling mechanism 230. In this way, the slip ring may electrically couple components (such as motor 205 and/or sensor(s) of blood pump 201) distal of coupling mechanism 230 (e.g., within blood pump 201) to component(s) (e.g., controller 142) proximal of coupling mechanism 230. An example of this aspect as implemented in assembly 200 is shown in FIG. 3B.

As shown in the example of FIG. 3B, coupling mechanism 230 is configured as through-hole slip ring and has portions 234 and 236. In this example, portions 234 and 236 may be generally tubular structure that are arranged concentrically as shown. Portion 234 is disposed rotatably within portion 236. In some aspects, portions 236 may be a “stationary portion” or stator of the slip ring and portion 234 may be a “rotating portion” or rotor of the slip ring. Portion 236 is attached to catheter 202, e.g., at distal end 203. Portion 234 is attached to proximal portion 213 of blood pump 201, e.g., at the proximal end thereof. As shown, in one aspect, the distal end of portion 234 projects distally from the distal end of portion 236. Portions 234 and 236 are configured to rotate relative to each other. For example, portions 234, 236 may be rotatably connected to each other by one or more bearings. In this way, the slip ring 230 is configured such that the blood pump 201 is rotatable relative to catheter 202.

The slip ring 230 is configured to provide and maintain constant electrical connection(s) between portions 234 and 236, even when portions 234 and 236 are rotated relative to each other. For example, as shown, portion 236 is configured to be coupled to one or more wires or conductors 262, 264, 266, and 268. Portion 234 is configured to be coupled to one or more wire or conductors 272, 274, 276, and 278. Portion 234 and the conductors 272-278 rotate together relative to portion 236 and conductors 262-268. Portion 236 may include a plurality of conductive brush contacts 292. Each brush contact 292 is configured to be electrically connected to a respective one of the conductors 262, 264, 266, and 268 coupled to portion 236. Portion 234 may include a plurality of conductive rings 294. Each ring 294 is configured to be electrically connected to a respective one of the conductors 272, 274, 276, and 278 coupled to portion 234. Each brush contact 292 is configured to be in constant contact with a respective ring 294 even when portion 234 is rotated relative to portion 236. For example, each brush contact 292 may be spring loaded or otherwise biased to contact a respective ring 294 to maintain contact. In this manner, an electrical connection or path is formed between each one of conductors 262, 264, 266, and 268 and a respective conductor 272, 274, 276, and 278. For example, in one aspect, via brush contacts 292 and rings 294, conductor 262 is electrically connected to conductor 272, conductor 264 is electrically connected to conductor 274, conductor 266 is electrically connected to conductor 276, and conductor 268 is electrically connected to conductor 278. The electrical connections between conductors 262-268 and 272-278 are maintained while portions 234, 236 are rotated relative to each other.

It is to be appreciated that the number of wires or conductors, brush contacts, conductive rings shown in FIG. 3B and described herein are exemplary and the slip ring 230 may be configured with more or less brush contacts and conductive rings than what is shown in FIG. 3B and described herein to electrically connect more or less wires or conductors.

In one aspect, conductors 262-268 may be electrically coupled to controller 142. For example, conductors 262-268 may extend through catheter 202 and plug 238 and be connected via cable 160 to controller 142. Moreover, conductors 272-278 may be electrically coupled to components of blood pump 201, such as motor 205 and/or one or more sensors 250. In this way, controller 142 may provide electrical power to and/or may communicate via electrical signals with the components of blood pump 201.

In one aspect, conductor 272 is coupled to sensor 250 (e.g., a pressure sensor or other type of sensor) and controller 142 is configured to communicate with sensor 250 (e.g., to receive measurement signals) via the electrical connection formed between conductor 272 and conductor 262. It is to be appreciated that although only one sensor 250 is shown in FIG. 3B, blood pump 201 may include a plurality of sensors each electrically connected to controller 142 by a different electrical path via slip ring 230 in the manner described herein.

Additionally, one or more of conductors 274-278 may be coupled to motor 205. For example, conductor 274 may be configured to receive electrical power provided by conductor 264 from controller 142 (or another power source). Moreover, conductor 276 may be configured as a ground to provide a return path to controller 142 (or another power source) via conductor 266. Thus, controller 142 may provide power to motor 205 to rotate the rotor or impeller 209 of blood pump 201. In some aspects, at least a third conductor 278 may be coupled to motor 205 to provide a data connection between controller 142 and motor 205 via conductors 278 and 268. The data connection (e.g., comprising digital or analog signals) may be used by controller 142 to control aspects of the operation of motor 205 (e.g., rotational speed/rotational direction that the motor rotates the rotor/impeller 209) and/or to receive data (e.g., related to the operation of motor 205) from motor 205.

As shown, in some aspects, slip ring 230 includes a through hole or bore 280 extending through the center of portion 234 from the proximal end to the distal end. The shaft 220 may extend through the bore 280. Additionally, the purge line and optical fiber(s) discussed herein may extend through bore 280 into blood pump 201, e.g., within shaft 220 or exterior to shaft 220.

It is to be appreciated that although slip ring 230 is shown in FIG. 3B with the “rotating” portion 234 connected to blood pump 201 and the “stationary” portion 236 connected to catheter 202, in other aspects of the present technology, the connections may be reversed. In this regard, the “rotating” portion 234 may be connected to catheter 202 and the “stationary” portion 236 may be connected to blood pump 201.

In any of the aspects described herein, assembly 200 may further comprise one or more fiber optic rotary joints or fiber optic slip rings configured to rotatably couple optical fibers of assembly 200 proximal and distal of the fiber optic rotary joints and to transmit optical communications between the optical fibers of assembly 200. For example, referring to FIG. 3C, a fiber optic rotary joint 295 is shown in accordance with aspects of the present technology. Fiber optic rotary joint 295 includes a distal portion 296 and a proximal portion 297. The proximal portion 297 is configured to rotate relative to distal portion 296. Moreover, the proximal portion 297 is configured to couple with one or more fibers 299 extending through catheter 202 and coupled, e.g., to controller 142 or another device or component. The distal portion 296 is configured to couple with one or more fibers 298 that are further coupled to one or more sensors. For example, fiber 298 may be coupled to an optical sensor 252 of blood pump 201 (e.g., disposed near outlet 206 and configured to sense blood pressure). Fiber optic rotary joint 295 is configured to transmit optical signals between one or more pairs of fibers 298, 299. Moreover, similar to slip ring 230, fiber optic rotary joint 295 is configured to permit fibers 299 and portion 297 to rotate relative to and fibers 298 and portion 296.

Fiber optic rotary joint 295 may be mounted within catheter 202 (e.g., at distal end 203 proximal of coupling mechanism 230), within coupling mechanism 230 (as described in relation to FIGS. 3A and 3B), within blood pump 201 (e.g., at proximal portion 213), within or to the distal end of shaft 220, or at any other location within assembly 200 to provide a rotary connection between optical fibers. The location of the fiber optic rotary joint 295 in assembly 200 is selected to reduce the torque applied to the optical fibers extending through catheter 202 when blood pump 201 is rotated relative to catheter 202. In one aspect, fiber optic rotary join 295 is mounted within the interior of the proximal portion 213 of blood pump 201 fibers 298 are coupled with sensors 250 and fibers 299 extend within shaft 220 or exterior to shaft 220 (but within catheter 202) toward the proximal end of catheter 202.

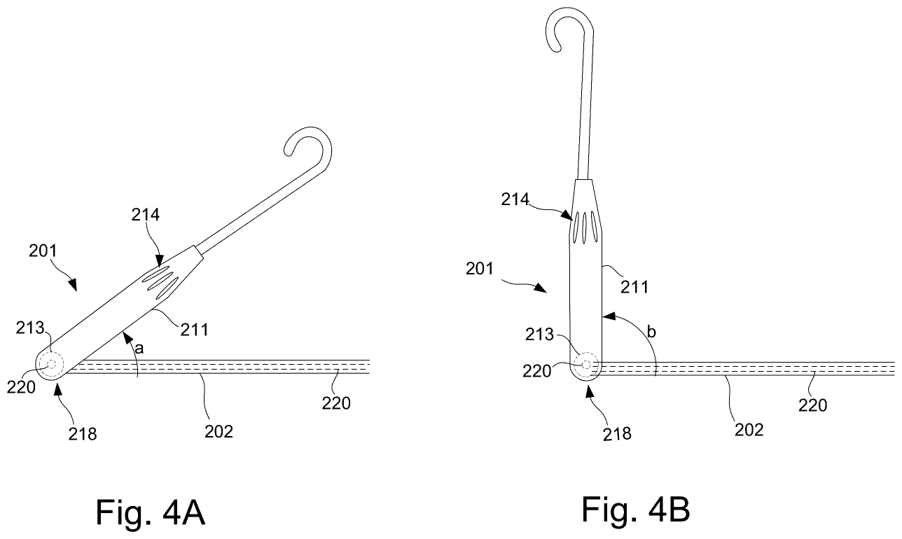

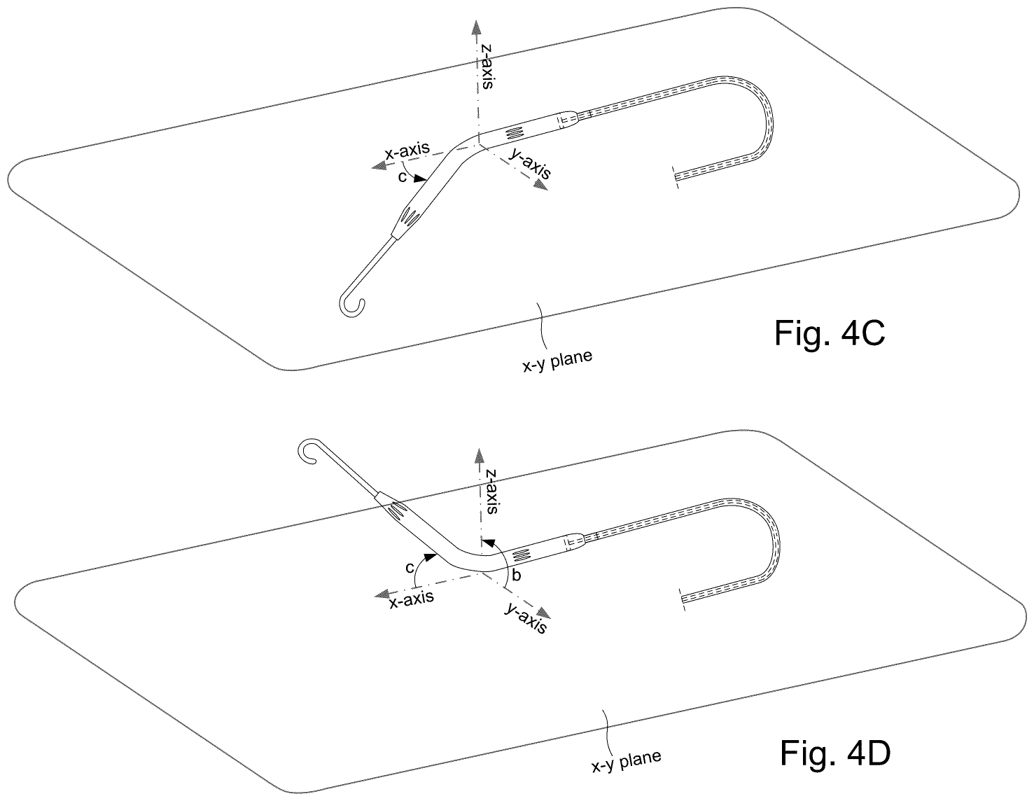

Referring now to FIGS. 4A-4D, rotation of blood pump 201 is shown in accordance with the present technology. As shown in FIGS. 4A-4D, in any of the aspects described herein, when shaft 220 is rotated, torque is transferred distally along the length of shaft 220 to the proximal portion 213 of blood pump 201 to rotate proximal portion 213 relative to catheter 202. It is to be appreciated that, in FIGS. 4C and 4D, the x, y, and z axes are orthogonal to each other and the x-axis is aligned with the proximal portion 213 of blood pump 201.

Due to the bend 218 between proximal portion 213 and distal portion 211, when the proximal portion 213 is rotated, the distal portion 211 is pivoted out of a two-dimensional x-y plane (shown in FIGS. 4C, 4D). At least the distal portion 203 of catheter 202 and the proximal portion 213 are disposed in the x-y plane when the assembly 200 is at rest and laying on a surface outside of the patient. As shown, distal portion 211 of blood pump 201 may be selectively pivoted to any one of a plurality of different positions out of the two-dimensional x-y plane based on the amount of torque the user applies to shaft 220 via steering mechanism 224. For example, as shown in FIG. 4A, when a first amount of torque is applied to shaft 220 by steering mechanism 224, the distal portion 211 of cannula 210 is pivoted by a rotational angle a out of the two-dimensional plane. Moreover, as shown in FIGS. 4B and 4D, when a second amount of torque that is greater than the first amount of torque is applied to the shaft 220 by steering mechanism 224, the distal portion 211 of blood pump 201 is pivoted by a rotational angle b out of the two-dimensional plane. Angle b is greater than angle a. Throughout the pivoting of distal portion 211 into and out of the x-y plane, the predetermined angle c between distal portion 211 and proximal portion 213 (measured from the x-axis to the distal portion 211) remains the same. However, the rotational angle of the distal portion 211 out of the x-y plane changes.

In one example, when the blood pump 201 and catheter 202 are deployed within the vasculature of a patient, the distal portion 211 of blood pump 201 is disposed within the left ventricle of the patient, the outlet 206 of the proximal portion 213 of the blood pump 201 is disposed in the ascending aorta, and at least a distal portion of the catheter 202 is disposed through the aorta. In this position, the shaft 220 may be torqued using steering mechanism 224 to rotate the blood pump 201 relative to the catheter 202 and thus pivot the distal portion 211, which carries the inlet 214, to a desired location and orientation within the left ventricle. For example, in one aspect, the desired orientation of blood pump 201 within the left ventricle comprises the distal portion 211 oriented to point toward the apex of the left ventricle such that the inlet 214 is not contacting any of the walls or other body structures (e.g., such as the mitral valve or the chordae that actuate the mitral valve) within the left ventricle to avoid a suction event between the inlet 214 and the tissues in the left ventricle. The coupling mechanism 230 (as described herein in relation to FIG. 3A or 3B) advantageously permits rotation (and torquing) of the blood pump 201 (and specifically proximal portion 213) relative to the catheter 202, without the catheter 202 being torqued or torsioned. Reducing or eliminating additional torques being applied to catheter 202 is beneficial for preventing kinking in catheter 202 or recoiling of catheter 202 if excessive torques are stored over the length of catheter 202. Reducing the torque applied to catheter 202 also reduces the stress applied to the wires and fibers extending through the inner lumen of catheter 202.

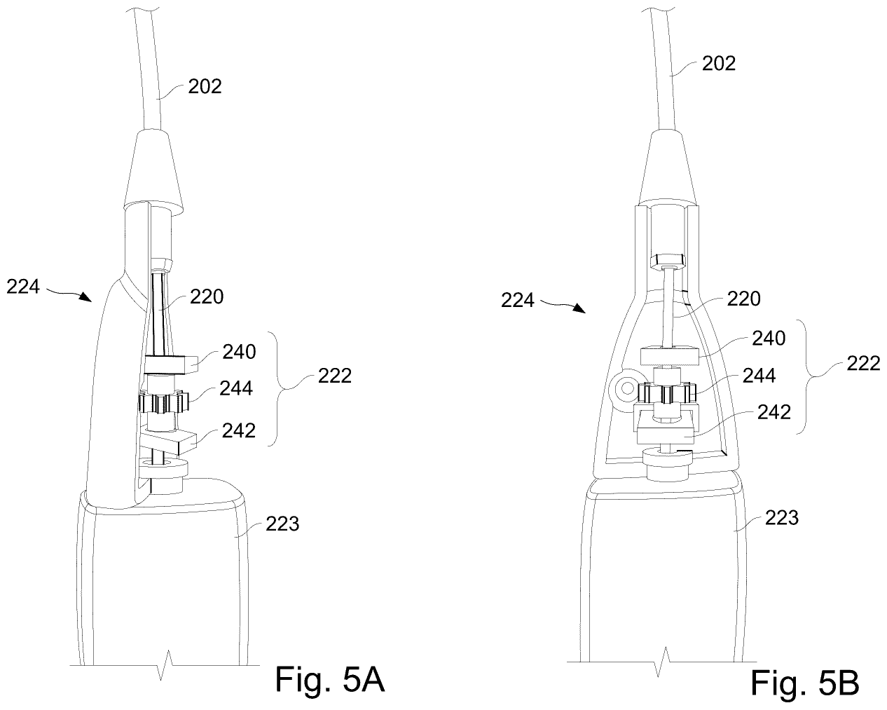

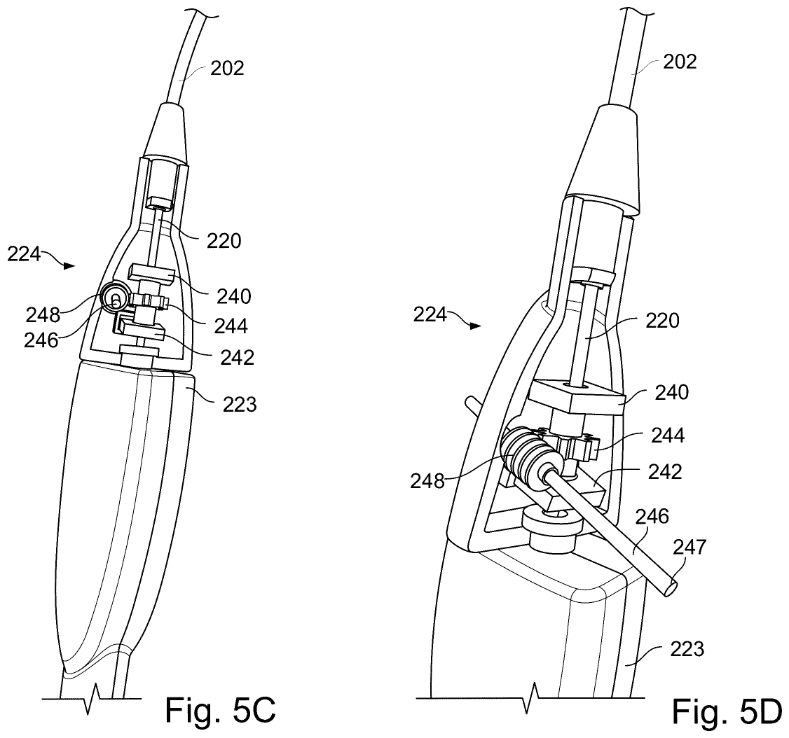

Referring to FIGS. 5A-5D, an exemplary implementation of steering mechanism 224 is shown in accordance with the present technology. It is to be appreciated that steering mechanism 224 is illustrated with a portion of the housing 223 removed to reveal certain components of steering mechanism 224 that may be disposed within housing 223. In the implementation of steering mechanism 224 shown in FIGS. 5A-5D, the steering mechanism 224 may be implemented in the plug 238 (e.g., as shown and explained in relation to FIG. 2C). Thus, housing 223 may be the housing for plug 238. However, in other aspects of the present technology, the implementation of the steering mechanism 224 shown in FIGS. 5A-5D may also be implemented as described above in relation to FIGS. 2D and 2E such that the steering mechanism 224 and housing 223 are a separate structure or component than plug 238.

In the implementation shown in FIGS. 5A-5D, the torque transfer mechanism 222 includes mounting members 240, 242, gear 244 (e.g., a spur gear or worm wheel), shaft 246, and gear 248 (e.g., a screw gear or worm gear). Gear 244 is fixedly mounted on a portion (e.g., a proximal portion) of shaft 220, such that shaft 220 extends through a lumen or channel of gear 244 and rotation of gear 244 causes a torque to be applied to shaft 220 to rotate shaft 220. Mounting member 240, 242 are rotatably coupled to respective portions of shaft 220 proximal and distal of gear 244. Mounting member 240, 242 are fixedly coupled to an interior of housing 223 to mount shaft 220 and gear 244 within the interior of housing 223. In this way, shaft 220 and gear 244 are rotatable relative to housing 223. Screw gear 248 is fixedly mounted to shaft 246 such that rotation of shaft 246 rotates screw gear 248. Shaft 246 is rotatably mounted to housing 223 such that the threads or teeth of screw gear 248 mate or engage with the teeth of gear 244. Thus, rotation of shaft 246 causes screw gear 248 to rotate, which in turn causes gear 244 to rotate and apply torque to shaft 220. In one aspect, shaft 246 is mounted to housing 223 such that an end, e.g., a portion or end 247 of shaft 246 extends exterior to housing 223. Exterior to the housing 223, the portion or end 247 is coupled to torque input mechanism 226, which is configured rotate shaft 246 to apply torque to shaft 220 and thereby steer cannula 210 of blood pump 201. The mechanism 226 may be a knob, dial, screw, thumbwheel, gear, etc. disposed on or near the exterior of housing 223, that a user manually turns or actuates to rotate shaft 246 and thus steer blood pump 201 and orient the distal portion 211 to a desired position relative to portion 213. Alternatively, the mechanism 226 may be a motor (e.g., a step motor) that is coupled to end 247 and controllable to rotate shaft 246. The motor may be controlled by the controller (e.g., controller 142) of assembly 200 via user input to the controller or by another controller integrated in or proximate to housing 223.

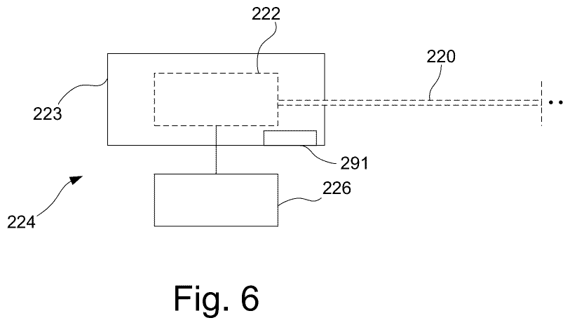

In any of the steering mechanism aspects described herein, a locking mechanism may be implemented with the steering mechanism that only permits the steering mechanism to be used to steer blood pump 201 when an authorized key is inserted into and unlocks the locking mechanism. For example, in one aspect, as shown in FIG. 6 a locking mechanism 291 may be implemented in housing 223. As described above, housing 223 may be the housing of plug 238 or may represent a distinct structure of assembly 200. Locking mechanism 291 is configured to disable the steering mechanism 224 from being operable unless an authorized key is received into a key hole of locking mechanism 291 and unlocks the locking mechanism 291. In one example, locking mechanism 291 may include a pin or other structures that is biased to interfere with one or both of the torque input mechanism 226 and/or the torque transfer mechanism 222 to prevent torque from being applied to drive shaft 220.

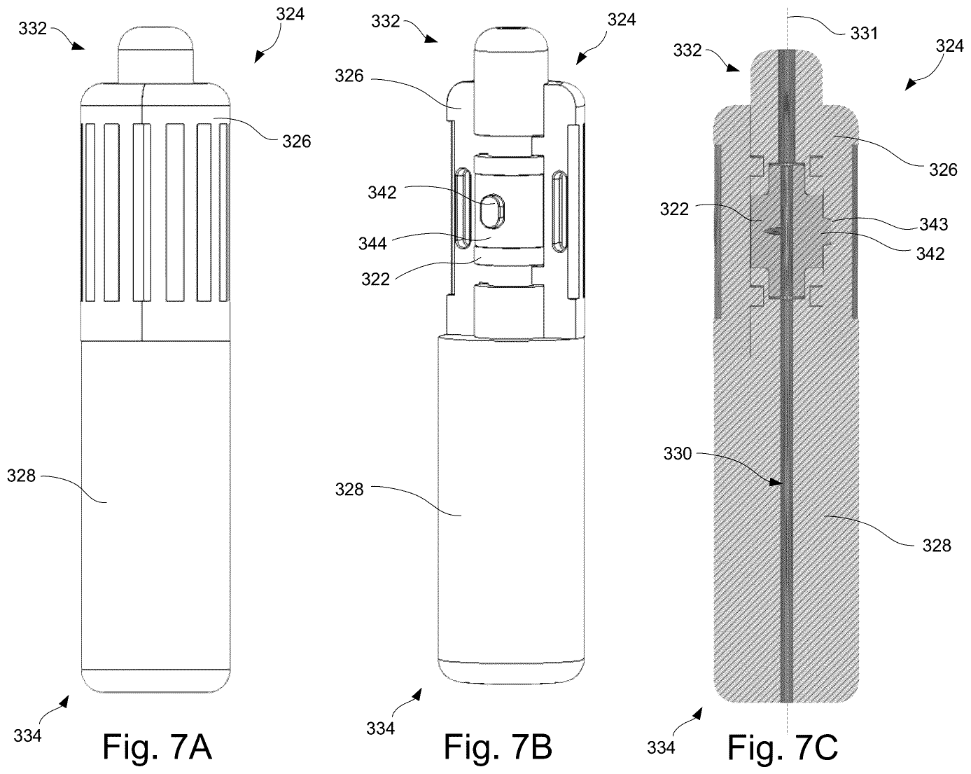

As described above in relation to FIGS. 2D and 2E, steering mechanism 224 may be implemented as a separate structure or component than plug 238 in accordance with the present technology. An exemplary implementation of the steering mechanism implemented as a separate structure than plug 238 is shown in FIGS. 7A-7C, which illustrate a steering mechanism or device 324 for torquing or rotating shaft 220. In one aspect, the steering mechanism 324 comprises an elongate, tubular body including a proximal end 332 and a distal end 334. Steering mechanism 324 comprises a rotatable member 326 that is rotatably mounted to a stationary member 328. As shown in FIG. 7B where a portion of rotatable member 326 is removed, a torque transfer member 322 is disposed within the interior of member 326. In one aspect, member 322 is cylindrically shaped and has one or more projections 342 that project from an exterior surface 344 of member 322. The projections 342 are received by corresponding slots 343 on an inner surface of member 326 such that when member 326 is rotated or torqued by a user, the torque is transferred to member 322 to rotate member 322. Steering mechanism 324 includes a lumen or channel 330 extending along a central axis 331 of steering mechanism 324 from proximal end 332 to distal end 334. Lumen 330 is formed in members 322 and 328 and is configured to receive a proximal portion of shaft 220. Member 322 is fixedly coupled to shaft 220. In this way, a user may grip and rotate or apply torque to member 326 to thereby rotate member 322 and thus shaft 220 to steer blood pump 201 in the manner described above. The user may also grip member 328 while gripping member 326 to achieve leverage and stability for rotating member 326.

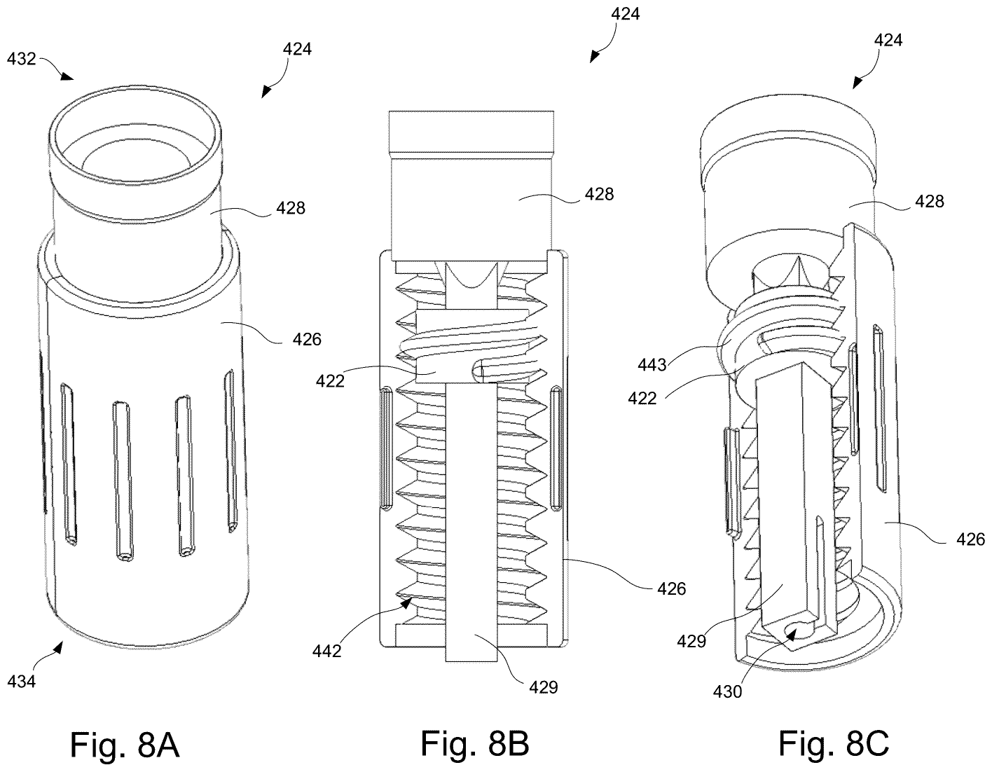

While the steering mechanisms of the present technology have been described as torquing the shaft 220, in other aspects of the present technology, steering mechanisms may be provided that push or pull the shaft 220 distally or proximally relative to catheter 202, which may change the tension in shaft 220 and also may change the orientation and/or shape of cannula 210 and blood pump 201. For example, referring to FIGS. 8A-8C, a steering mechanism 424 is shown in accordance with the present technology. The steering mechanism 424 includes ends 432, 434 and members 422, 426, 428. Member 426 is rotatable relative to member 428. Member 428 includes a channel or lumen 430 configured to receive a proximal portion of shaft 220. The exterior surface of member 426 is configured to be gripped and rotated by a user. The interior surface of member 426 includes screw threads 442. Member 422 is mounted to an elongate shaft 429 of member 428 that extends within the interior of member 426. Member 422 includes outwardly projecting screw threads 443. When member 426 is rotated by a user, screw threads 442 cooperate with screw threads 443 to translate the torque inputted to member 426 by a user into longitudinal pushing or pulling of shaft 220.

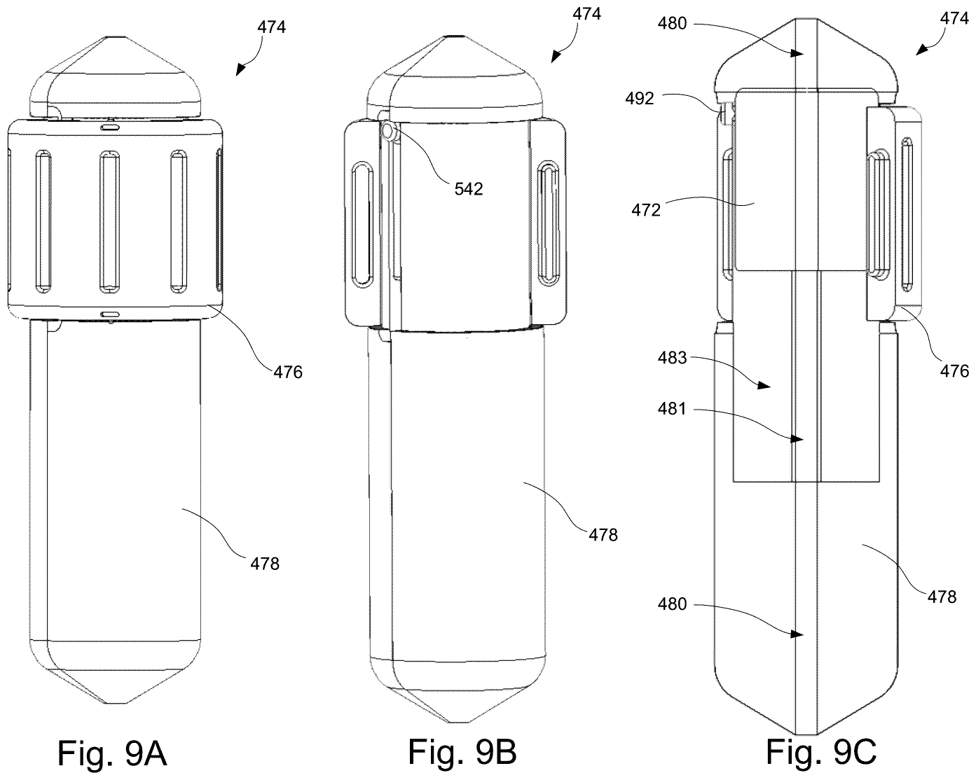

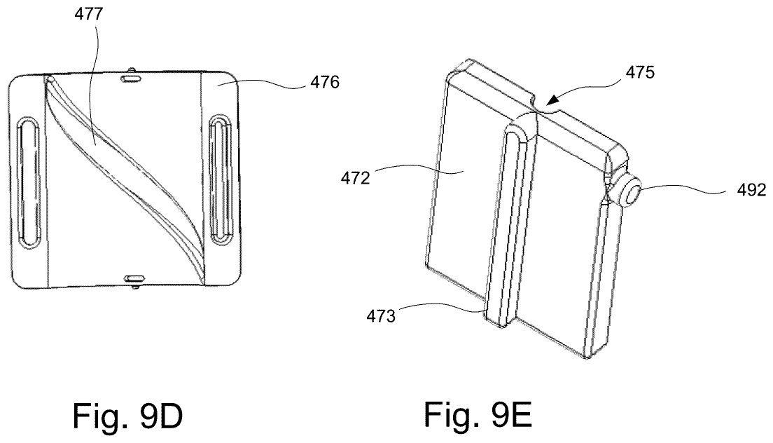

FIGS. 9A-9C illustrate another steering mechanism 474 configured to push or pull shaft 220. FIGS. 9D and 9E illustrate members 472 and 476 of mechanism 474. The securing mechanism 474, in some aspects, may have an elongate tubular shape and includes a member 476 that is rotatably mounted on a stationary elongate member 478. Within the interior of member 478 a member 472 is slidably mounted such that the member 472 can slide longitudinally (i.e., proximally and distally) relative to member 478. For example, the interior of member 478 includes compartment 483 configured to slidably receive member 472 and confine the motion of member 472 to longitudinal motion (proximal and distal motion). The compartment 483 includes a slot 481 that receives a longitudinal projection 473 of member 472 to guide the longitudinal motion of member 472 within compartment 483 and relative to stationary member 478. The interior of member 478 also includes a slot 480 (formed distal and proximal of compartment 483) that is configured to slidably receive a proximal portion of shaft 220. Member 472 includes a projection 492 that extends into a helical slot 477 on an inner surface of member 476. When member 476 is rotated relative to member 478 in a first direction (e.g., clockwise), the projection 492 is guided by helical slot 477 to force the member 472 to slide in a first direction (e.g., proximally). Alternatively, when member 476 is rotated relative to member 478 in an opposite direction (e.g., counterclockwise), the projection 492 is guided by helical slot 477 to force the member 472 to slide in a second direction (e.g., distally). The shaft 220 is fixedly coupled to the member 472 (e.g., partially received in slot 475) such that when member 472 is pushed distally relative to member 478, the shaft 220 is pushed distally, and when member 472 is pulled proximally relative to member 478, the shaft 220 is pulled proximally.

It is to be appreciated that, in some aspects, steering mechanisms 424 and 474 may, in addition to pulling and pushing, be configured to torque shaft 220.

Steering mechanisms 324, 424, 474 may be implemented in assembly 200 in the manner described above in relation to FIG. 2D or 2E. In this regard, steering mechanisms 324, 424, 474 may be coupled to shaft 220 via a sidearm 261 of plug 238 (as shown in FIG. 2D) or may be coupled to shaft 220 distal of plug 238 (as shown in FIG. 2E). Alternatively, steering mechanisms 324, 424, 474 may replace plug 238 or be integrated with plug 238.

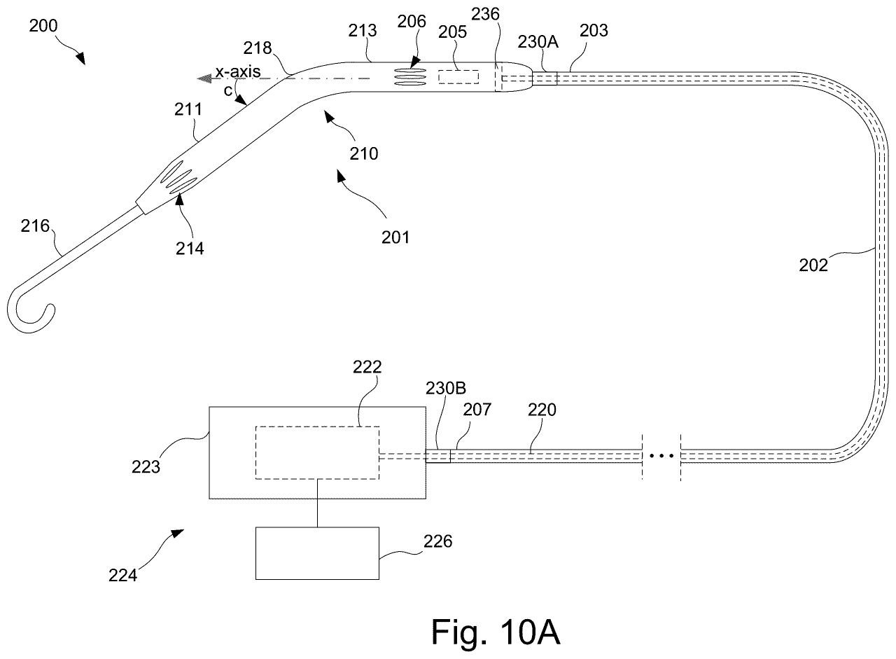

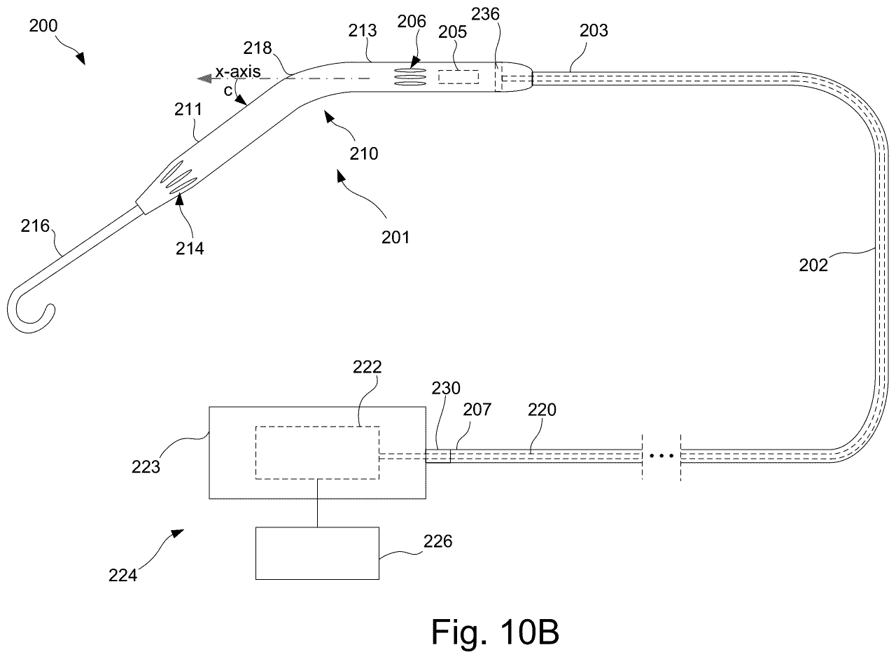

In some aspects, assembly 200 may include multiple coupling mechanisms 230 (configured as described in relation to FIG. 3A or FIG. 3B). For example, referring to FIG. 10A, assembly 200 is shown with coupling mechanisms 230A, 230B, which are coupled at distal and proximal ends of catheter 202. Coupling mechanisms 230A, 230B may include any of the features described above in relation to FIGS. 3A and 3B. As described, coupling mechanisms 230 may be rotary joints, such as slip rings, each with a through hole or bore that permits passage of drive shaft 220 and/or wires, purge lines, optical fibers, etc. of assembly 200 through the through hole. An advantage to arranging coupling mechanisms 230A, 230B at each of the ends of catheter 202 is that the torsion and torque on catheter 202 is greatly reduced because on each end of the catheter 202 the distal and proximal components, such as pump 201, plug 238, and/or steering mechanism 224, are freely rotatable relative to catheter 202. It is to be appreciated that although coupling mechanism 230B is shown coupled directly to housing 223 of steering mechanism 224, in other aspects, as described above, the coupling mechanism 230B may be coupled directly to plug 238, for example, in a similar arrangement as shown in FIG. 2D.

In another aspect, assembly 200 may include the coupling mechanism 230 (configured as described in relation to FIG. 3A or FIG. 3B) only at the proximal end of the catheter 202, as shown in FIG. 10B. In the aspect shown in FIG. 10B, the proximal portion 207 is freely rotatable relative to the component it is coupled to, e.g., the steering mechanism housing 223, as shown, or to plug 238 (not shown). In this way, when blood pump 201 is steered using steering mechanism 224, the torque in catheter 202 may be released at end 207.

In any of the aspects described herein, the assembly 200 may be configured to limit the amount of rotation that steering mechanism 224 imparts onto blood pump 201 to prevent overly torquing or stressing wires, fibers, purge line, etc. disposed through catheter 202. In some aspects, the steering mechanism 224 and/or coupling mechanism(s) 230 may include stop(s) configured to stop further rotation past a predetermined amount in each rotational direction. In aspects where steering mechanism 224 is a slip ring (e.g., FIG. 3B), the range of motion of each slip ring may be limited to predefined safe ranges or limits (e.g., 270 degrees rotation, 360 degrees rotation, or any other suitable range or limit).

In some aspects, the wires, fibers, and/or purge line may each have a predetermined amount of “slack” (i.e., a greater length than required to make the necessary connections) so that the wires, fibers, and/or purge line can absorb a certain amount of rotation or torque without being damaged.

In aspects where one or more coupling mechanisms 230 comprise a slip ring, the electrical wires within catheter 202 (e.g., for the sensors, pump motor, communications, etc.) may be electrically coupled through the slip ring. In this way, torquing and damage to the wires in catheter 202 and to the catheter 202 is reduced even when pump 201 is rotated.

In any of the aspects described herein, as described above assembly 200 may further include one or more fiber optic rotatory joints, e.g., near one or both ends of catheter 202 or at any other point along the fiber (or fibers) in catheter 202 to reduce or eliminate torques on the fibers in catheter 202 even when pump 201 is rotated.

In any of the aspects described herein, a coupling mechanism, such as coupling mechanism 230 (e.g., a slip ring) may be added between cable 160 and plug 238 to permit cable 160 and the proximal end of plug 238 to rotate freely (or within predefined limits) relative to each other. This will further reduce unwanted torque and strain on catheter 202.

In any of the aspects described herein, assembly 200 may be configured to provide some indications to the user of the amount that blood pump 201 has been steered relative to a starting position or how much torque has been applied to shaft 220. For example, in one aspect, the torque input mechanism 226 (e.g., a knob, dial, etc.) includes markings that indicate to the user how much torque is applied to shaft 220 and/or how much blood pump 201 has been rotated relative to a starting position.

In any of the aspects described herein, assembly 200 may include one or more sensors configured to detect the amount that blood pump 201 has been steered relative to a starting position or how much torque has been applied to shaft 220. The one or more sensors may be included in the steering mechanism, at a point along shaft 220, in blood pump 201, etc. Where a motor is used to rotate shaft 220, controller 142 may detect the amount of rotation by detected the amount the motor shaft has been rotated or the amount of current drawn within a period of time corresponding to the rotation.

In any of the aspects described herein, assembly 200 may be modified to include multiple steering or actuating mechanisms. For example, as described above, the steering mechanisms of the present technology may torque the blood pump 201. In some aspects, the assembly 200 may further including a second steering mechanism for bending the catheter 202 at one or more points (e.g., to selectively match the geometry of the patient's anatomy during when assembly 200 is inserted into the patient) or changing the bend angle c between portions 211 and 213 of cannula 210. Each steering mechanism may have a different steering effect on the assembly 200. Moreover, any of the steering mechanisms described herein may be modified to perform multiple types of steering/actuation. For example, steering mechanism 224 may be modified to both torque shaft 220, push/pull shaft 220, and/or cause bending to occur in catheter 202 or pump 201.

As described above, in assemblies, such as assembly 100 that do not have a steering mechanism, such as steering mechanism 224, to alter the position of the pump 101 within the patient, a user may attempt to grasp the portion 181 (shown in FIG. 1C) of the catheter 102 that is exterior to the patient and apply torque to the catheter 102 to torsion/twist the catheter 102 and thereby rotate the cannula 110 to change the orientation of distal cannula 111 within the patient's heart. Moreover, the user may pull or push the catheter 102 into or out of the patient. However, the user may find that there is no available stable position in portion 181 of catheter 102 or any other portion of assembly 100 exterior to the patient to sufficiently grip and manually manipulate the catheter 102 in order to manually steer the blood pump 101.

As described herein, the inventors have recognized the benefits of improving a user's ability to apply torque to a catheter of a catheter assembly and adjust a position of the blood pump at the distal end of the catheter. The present technology provides devices and assemblies with features that enhance the user's ability to grip the portions of the pump assembly (such as portion 181 of catheter 102) that are exterior to the patient to give the user sufficient control to steer pump 101 by torquing, pushing, or pulling the catheter 102.

In some aspects of the present technology, a securement device, such as any of the securement devices (e.g., device 132 or any other type of securement device) described herein, may be configured to enable the user to grip portion 181 of the catheter 102 with sufficient friction, force, and/or stability to torque, push, and/or pull catheter 102 and as needed by a clinician to steer blood pump 101 to a desired position within the patient.

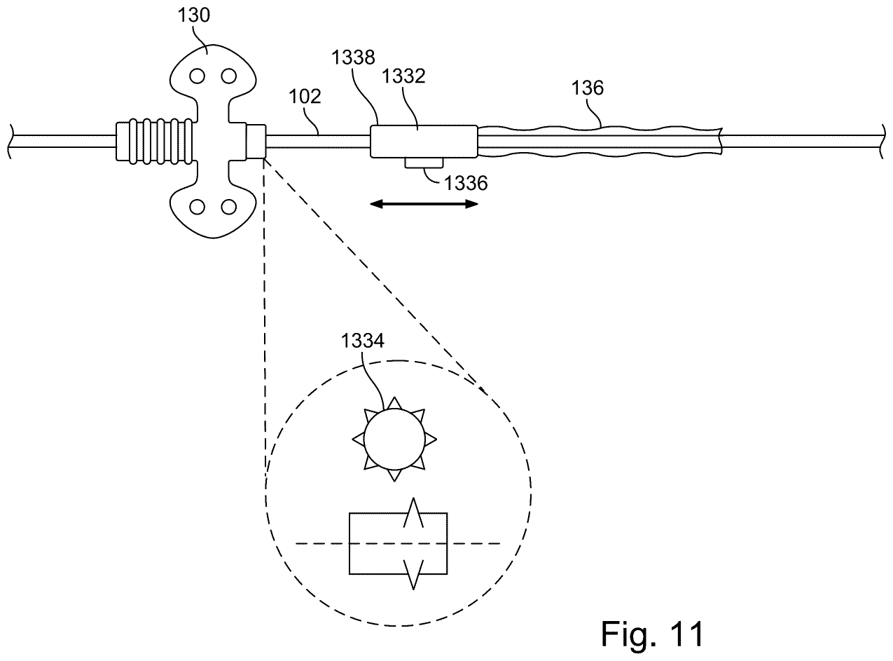

For example, as shown in FIG. 11, a securement device 1332 is provided in accordance with one aspect or embodiment of the present technology. It is to be appreciated that securement device 1332 may include any of the features of securement devices described herein. In this regard, the securement device 1332 may include a securing mechanism configured to be selectively engaged (e.g., by pressing a button or turning a barrel) to restrict or permit the movement of an object (e.g., catheter 102) inserted through the securement device 1332. For example, the securing mechanism may comprise a Tuohy-Borst device with a rotatable barrel (such as barrel 132c) as used in securement device 132 to vary the amount of resistance imposed on the catheter 102 inserted through the securement device. Alternatively, the securing mechanism may comprise a spring-loaded button (e.g., as used in securement devices described in U.S. Patent Application Publication No. 2022/0032037 that compresses a flexible sleeve to resist the movement of the catheter 102 through the securement device).

When the securing mechanism of securement device 1332 is not engaged (and thus not restricting the movement of catheter 102), the securement device 1332 may be configured to be slidable or otherwise displaceable over catheter 102 (e.g., along portion 181). In this regard, the securing device may be permanently attached to the catheter (e.g., at portion 181). In other embodiments, the securing device may be an accessory that is attachable to the catheter (e.g., at portion 181) to allow a clinician to move the catheter and, thus, pump, into a desired location and then remove the securing mechanism when not in use.

In some embodiments, the length of securement device 1332 from its proximal to distal end may be selected to enable the user to easily grip the exterior of securement device 1332 in a stable manner. For example, in some embodiments, the length of the device 1332 may be between 1 and 6 inches, although it will be appreciated by the skilled person that other suitable ranges may be selected depending upon the configuration of the device.

Securement device 1332 may include a button, slider, thumbwheel, collar or other actuating member 1336 for actuating the securing mechanism of securement device 1332 to internally grip the exterior of catheter 102. In some embodiments, the device may also include a separate gripping mechanism for gripping catheter 102 that is separate from the securing mechanism (i.e., spring-loaded button), such as that described above. The actuating member 1336 may be selectively engaged by the user to cause the securement device 1332 to grip the exterior of the catheter 102 inserted through device 1332. With the catheter 102 gripped by securement device 1332, the user may grasp the exterior of securement device 1332 and push, pull, and/or torque the securement device 1332 to thereby push, pull, or torque the catheter 102 to position the blood pump 101 appropriately within the patient. In one aspect, when actuating member 1336 is engaged by the user, an inner diameter of a component of the securing mechanism of securement device 1332 is reduced to grip the exterior of catheter 102.

In one aspect, the proximal end of the butterfly 130 may include an attachment member 1334 (e.g., a magnet or mechanical coupling member) that engages and attaches to a corresponding attachment member 1338 (e.g., a magnet or mechanical coupling member) at the distal end of securement device 1332. In this way, the butterfly 130 may be securely coupled to securement device 1332. Attachment members 1334, 1338 may be configured to automatically attach to each other when manually brought together by a user (e.g., by way of a snap fit connection, magnetic connection, or any other suitable connection). In some aspects, securement device 1332 may include a second actuating member (not shown) accessible on the exterior of securement device 1332. The second actuating member may be configured to change between different states or positions. For example, in some embodiments, a first state or position may cause members 1334, 1338 to attach when members 1334, 1338 are brought into contact with each other. A second state or position may cause members 1334, 1338 to detach from each other. The second actuating member may be a button, knob, slider, etc., or other means to control the attachment or detachment of members 1334, 1338.

In one aspect, the securement device 1332 may be coupled to the sleeve 136. For example, as shown in FIG. 11, the proximal end of securement device 1332 may be coupled to the distal end (or a distal portion) of sleeve 136. In some aspects, securement device 1332 may be coupled, attached, or otherwise joined (e.g., using adhesives, retaining rings, or other suitable techniques) to the sleeve 136.

In one aspect, actuating member 1336 may be actuated by a user to achieve a locked state. In the locked sate, without further user engagement (e.g., without the user applying continued force to engage actuating member 1336), the securement device 1332 may continue gripping catheter 102 until the actuating member 1336 is actuated by a user to achieve an unlocked state. As will be appreciated, in embodiments in which the actuating member is fixedly attached to the catheter, it may remain connected to the catheter whether or not in the locked state.

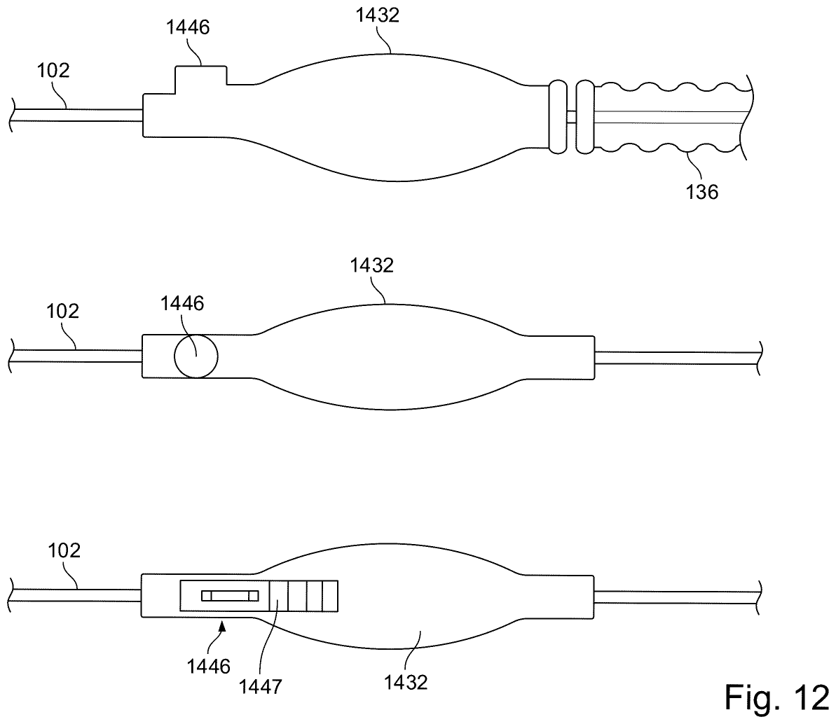

Referring to FIG. 12, another securement device 1432 that is configured to grip the exterior of catheter 102 to permit the user to push, pull, and/or torque the catheter 102 is illustrated in accordance with the present technology. It is to be appreciated that securement device 1432 may include any of the features of securement devices described herein. In this regard, the securement device 1432 may include a securing mechanism configured to be selectively engaged to restrict or permit the movement of an object (e.g., catheter 102) inserted through the securement device 1432. For example, the securing mechanism may comprise a Tuohy-Borst device with a rotatable barrel (such as barrel 132c) as used in securement device 132 to vary the amount of resistance imposed on the catheter 102 inserted through the securement device. Alternatively, the securing mechanism may comprise a spring-loaded button that compresses a flexible sleeve to resist the movement of the catheter 102 through the securement device.

In any case, securement device 1432 may have a housing with an exterior surface that has an enlarged ergonomic shape and a length (measured from proximal to distal) that permits a user to easily grip the exterior of securement device 1432 and manually push, pull, and/or torque securement device 1432 with a single hand. As shown, the ergonomic shape of the exterior may include a convex surface. In one aspect, securement device 1432 includes a button, slider, knob, or other actuating member 1446 to actuate the securing mechanism of securement device 1432 to internally grip the exterior of catheter 102. As with other embodiments, the securing device 1432 also may include a gripping mechanism for gripping catheter 102 that is separate from the securing mechanism (e.g., spring-loaded button). The actuating member 1446 may be selectively engaged by the user to cause the securement device 1432 to grip the exterior of the catheter 102 inserted through device 1432. With the catheter 102 gripped by securement device 1432, the user may grasp the exterior of securement device 1432 and push, pull, or torque the securement device 1432 to thereby push, pull, or torque the catheter 102 to position the blood pump 101 appropriately within the patient. In one aspect, when actuating member 1446 is engaged by the user, an inner diameter of a component of the securing mechanism of securement device 1432 may be reduced to grip the exterior of catheter 102. In one aspect, the proximal end of securement device 1432 may be configured to be removably coupled or connected with sleeve 136. In one aspect, the securement device 1432 may be integrated with the repositioning unit 180.

In one aspect, actuating member 1446 comprises a lockable slide 1447. In a first position of the slide 1447, the securement device 1432 does not grip the exterior of the catheter 102. Instead, the actuating member 1446 may be moveable relative to the catheter when the slide is in the first position. In some embodiments, the lockable slide 1447 may be configured to lock when pushed to a second position of the slide 1447. In some embodiments, the slide is configured such that a user may maintain contact with the slide to keep the slide in the locked position. In other embodiments, the lockable slide may auto-lock with a low application of force from the user. When the slide 1447 is auto-locked, user contact with the slide 1447 can be discontinued and the slide 1447 may stay in the second position. In the second position, the slide 1447 causes the securement device 1442 to continuously grip the exterior of catheter 102 unless the user pushes slide 1447 back to the first position. If the user applies additional force to slide 1447, which causes slide 1447 to move to a third position, the securement device 1432 may be configured to apply an increased force (relative to the force applied in the second position) to the exterior of catheter 102, which allows the user to apply greater torque, pushing, or pulling force to catheter 102 through device 1442. In one aspect, advancing the slide 1447 to the opposite end of the slot or track causes the securement device 1442 to detach from butterfly 130.

It is to be appreciated that, in some aspects, securement devices 1332, 1432 may be configured to include a mechanism for applying torque to catheter 102 once gripped by securement devices 1332, 1432. For example, securement devices 1332, 1432 may include a thumbwheel, gear, knob, or other type of mechanism that is accessible to the user on an exterior surface of devices 1332, 1432 and that the user can actuate. The mechanism may be configured to transfer the torque applied by the user to catheter 102, thereby torquing catheter 102 and steering the pump 101.

In some aspects of the present technology, a securement device, such as any of securement devices described herein may be provided with a grippable handle configured to enable the user to grip portion 181 of the catheter 102 with sufficient friction, force, and stability to torque, push, and/or pull catheter 102 accurately so that the user may steer blood pump 101 as desired within the patient.

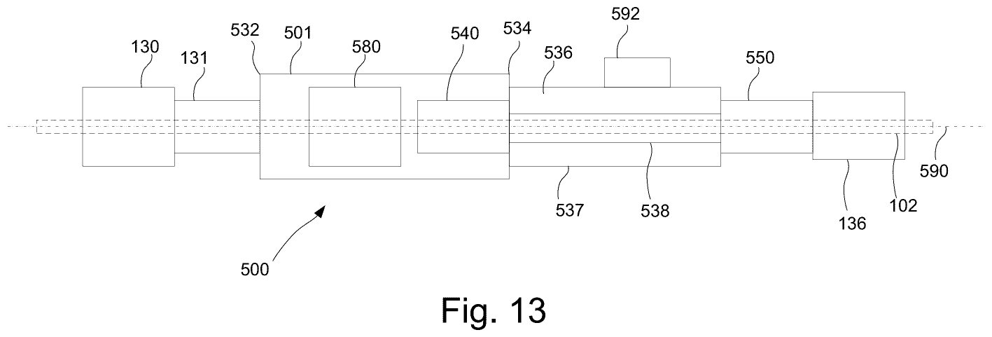

For example, FIG. 13 illustrates a securement device 500 provided with a grippable handle 536 in accordance with the present technology. Securement device 500 may include any of the features described herein in relation to securement devices.

In one aspect, the securement device 500 includes a housing 501 including a distal end 532 and a proximal end 534. The housing 501 may extend along a longitudinal axis 590 from the proximal end 534 to the distal end 532. The distal end 532 of the securement device 500 may be configured to couple with a fixation device 130 (e.g., a butterfly fixation device) that is attachable to the skin of the patient proximate to the access site for the assembly 100. In some aspects, distal end 532 includes a cavity configured to receive a proximal end of a hemostasis valve 131 that couples the securement device 500 to the fixation device 130. In some aspects, the fixation device 130 or the securement device 500 may be integrated with the hemostasis valve 131 and the distal end 532 of the securement device 500 couples directly with the fixation device 130.

The securement device 500 may further include a securing mechanism 580 such as any of the securing mechanisms described herein. The securing mechanism 580 may be configured to be selectively engaged by the user to restrict or permit the movement of an object (e.g., catheter 102) inserted through housing 501 along axis 590. For example, the securing mechanism 580 may comprise a Tuohy-Borst device with a rotatable barrel (such as barrel 132c) as used in securement device 132 to vary the amount of resistance imposed on the catheter 102 inserted through the securement device 500. Alternatively, the securing mechanism 580 may comprise a spring-loaded button that compresses a flexible sleeve to resist the movement of the catheter 102 through the securement device 500.

The securement device 500 further includes a grippable handle 536. The grippable handle 536 includes a proximal end, a distal end, an exterior surface 537, and an interior surface defining an interior lumen 538. The grippable handle 536 and the interior lumen 538 each may extend from their respective proximal to distal ends along the longitudinal axis 590. The interior lumen 538 may be configured to receive a portion of the catheter 102 that is exterior to the patient, e.g., portion 181, described above. In some aspects, the diameter of lumen 538 may be configured to be larger than the outer diameter of catheter 102 when the grippable handle 536 is in an unstressed state and free of externally applied forces. Thus, when the securing mechanism 580 is not engaged to restrict the movement of catheter 102 through housing 501 and distal end 532 is not coupled to fixation device 130, the securement device 500 is slidable over catheter 102 along the longitudinal axis 590.

In some embodiments, the distal end of the grippable handle 536 may be rotatably coupled to the proximal end 534 of housing 501 such that grippable handle 536 is rotatable relative to housing 501. The grippable handle 536 may be configured to be deformable in response to a compression force applied to the grippable handle 536. In some aspects, the grippable handle 536 may made of rubber, silicone, or another suitable material that is deformable. The compression force may be applied by a user gripping the exterior surface 537 of handle 536 while the user is applying the torque (or other movement) to move the catheter and, thus, the pump to the desired location in the patient's body. Alternatively, in some aspects, securement device 500 may include an actuator 592 (e.g., a slider, knob, thumbwheel, or other actuator) configured to apply a compression force to grippable handle 536 when actuated by a user. The actuator 592 may be configured to be lockable (e.g., as described above in relation to slide 1447) to maintain the compression force on grippable handle 536 until the actuator 592 is unlocked by the user.

When the grippable handle 536 is deformed, at least a portion of the internal lumen 538 of grippable handle 536 may deform such that the interior surface forming the lumen 538 grips the exterior of catheter 102. In this way, when the compression force is applied to grippable handle 536 and the catheter 102 is gripped, the user may advantageously torque the grippable handle 536, which transfers the torque to the gripped portion of the catheter 102 to permit the user to position the blood pump 101 appropriately within the patient. In some aspects, the exterior surface 537 of grippable handle 536 may have an ergonomic shape to aid in the user's ability to comfortably grip and apply compression to the grippable handle 536. The grippable handle 536 has a sufficient length from its proximal end to its distal end to permit a stable grasping of grippable handle 536 and the catheter 102. For example, in some embodiments, the handle may be between 1 and 6 inches long, although the handle may have other suitable lengths.

In some aspects, the grippable handle 536 is configured such that, after the compression force is removed (i.e., is no longer being applied by the user) to grippable handle 536, the grippable handle 536 (and the interior lumen 538) substantially returns to its original unstressed state.

In some aspects, securement device 500 may include a coupling mechanism 540 configured to rotatably couple the grippable handle 536 to the housing 501. In some aspects, the coupling mechanism 540 is further configured to permit the grippable handle 536 to be translated along the longitudinal axis 590 (proximally or distally) relative to housing 590. Thus, when the compression force is applied to grippable handle 536 and the catheter 102 is gripped, the user may advantageously push or pull the grippable handle 536, which transfers the push or pull force to the gripped portion of the catheter 102 to permit the user to position the blood pump 101 appropriately within the patient. In this way, securement device 500 permits the user to simultaneously rotate and longitudinally translate catheter 102 using handle 536.

In some aspects, securement device 500 may include a second coupling mechanism 550 configured to rotatably couple the grippable handle 536 to the protective sleeve 136 such that the grippable handle 536 is rotatable relative to the protective sleeve 136. In this way, the protective sleeve 136 is not torqued when the grippable handle 536 is torqued. In some embodiments, this may prevent sleeve 136 from being damaged by the rotation of handle 536.

As discussed herein, the features of securement device 500 that aid in gripping and positioning the catheter 102 may be implemented with any of the securement devices described herein.

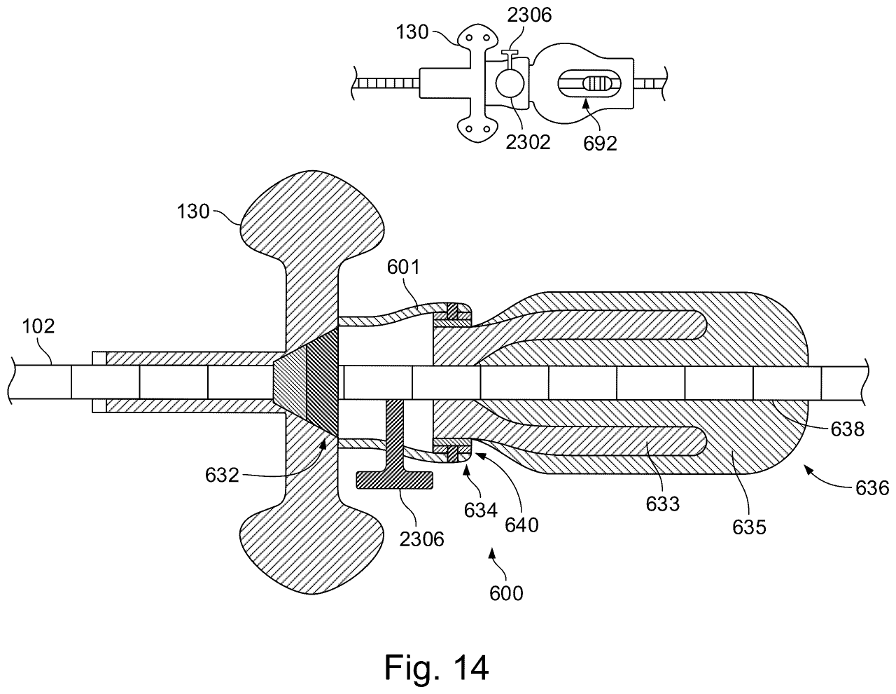

For example, referring to FIG. 14, a securement device 600 that may include a grippable handle 636 is shown in accordance with the present technology. Securement device 600 may include a housing 601 with a distal end 632 and a proximal end 634. The housing 601 may be configured to use the securing mechanisms described above in in U.S. Patent Application Publication No. 2022/0032037 for selectively restricting the movement of catheter 102 when inserted through housing 601. In this regard, securement device 600 may include a flexible sleeve (not shown), button 2302, spring, and retention pin 2306 for restricting the movement of catheter 102. However, in the aspect shown in FIG. 14, the proximal end 634 of housing 601 may configured to be coupled with a grippable handle 636 such that grippable handle 636 is rotatable relative to housing 601. In one aspect, handle 636 may be rotatably coupled to housing 601 via a coupling mechanism 640. The coupling mechanism 640 may include bearings configured to permit handle 636 to rotate relative to housing 601.

The distal end 632 of housing 601 may be configured to couple with a fixation device 130. In some aspects, the distal end 632 may include a cavity configured to receive a proximal portion of hemostasis valve 131 and to couple the housing 601 with fixation device 130.

Handle 636 may include any of the features of handle 536 described above. In this regard, handle 636 may include an interior lumen 638 configured to receive a portion of catheter 102. Moreover, handle 636 may be deformable in response to a compression force applied to handle 636. The compression force may be applied manually by a user gripping the exterior surface of handle 636 and squeezing. Alternatively, the compression force may be applied using an actuator 692 (e.g., a button, slider, thumbwheel, etc.) configured to permit the user to selectively apply compression force to handle 636. In one aspect, the actuator 692 is a slider. The slider may be configured to be biased to a first position (e.g., by a spring) such that in the first position the slider does not apply any compression force to handle 636. The slider may be advanced to a second position such that in the second position the slider applies compression force to handle 636 to deform handle 636 such that the interior surface of handle 636 defining lumen 638 grips the catheter 102. The slider may be configured to permit a user to lock the slider in the second position such that the user's hand is free to rotate handle 636 to apply torque to catheter 102.

In one aspect, handle 636 comprises a first portion 635 and a second portion 633. The second portion 633 has a higher material hardness and is less deformable than the first portion 635. The hardness of the second portion 633 is sufficiently high to aid handle 636 in maintaining its structural integrity and to provide secure coupling to housing 601 via coupling mechanism 640. The first portion 635 is configured to be soft enough to deform in response to a compression force applied to the portion 635 to grip catheter 102 so that when a user torques the handle 636, the torque is transferred and applied to catheter 102 to steer and position pump 101.

As will be appreciated, the housing portion that engages with the handle and connects to the butterfly and the handle (e.g., a housing collar) may have any suitable cross-sectional shape. For example, the housing portion may be cylindrical, triangular, polygonal or other suitable shape.

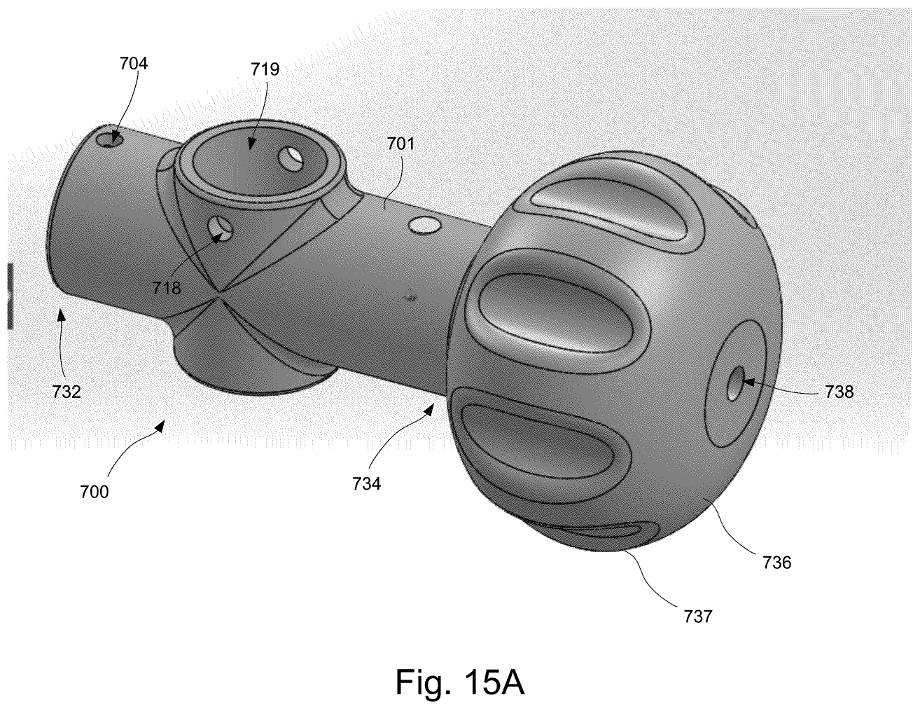

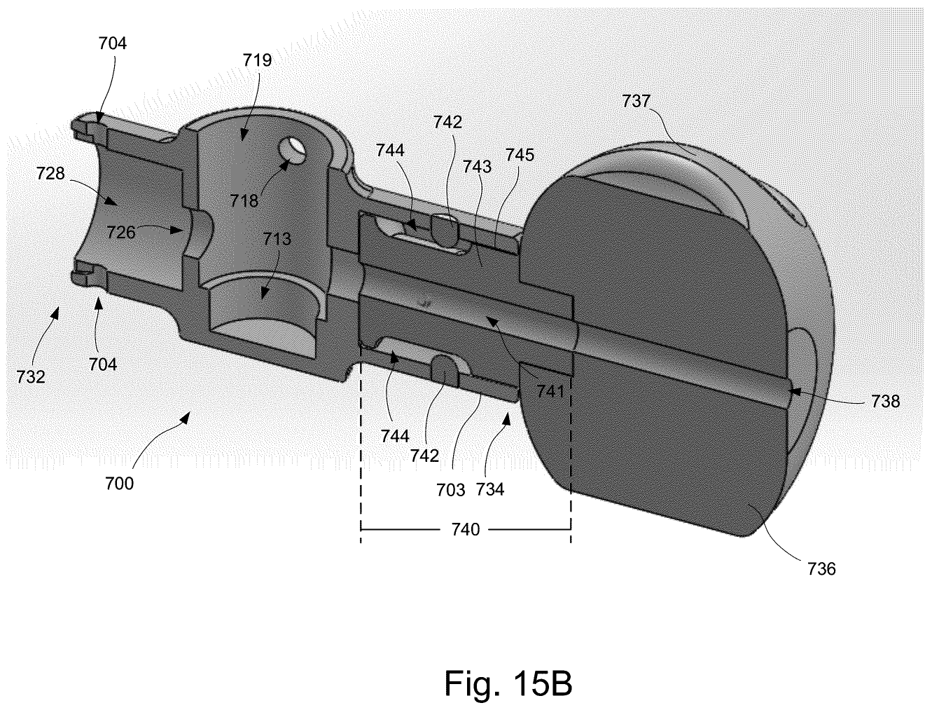

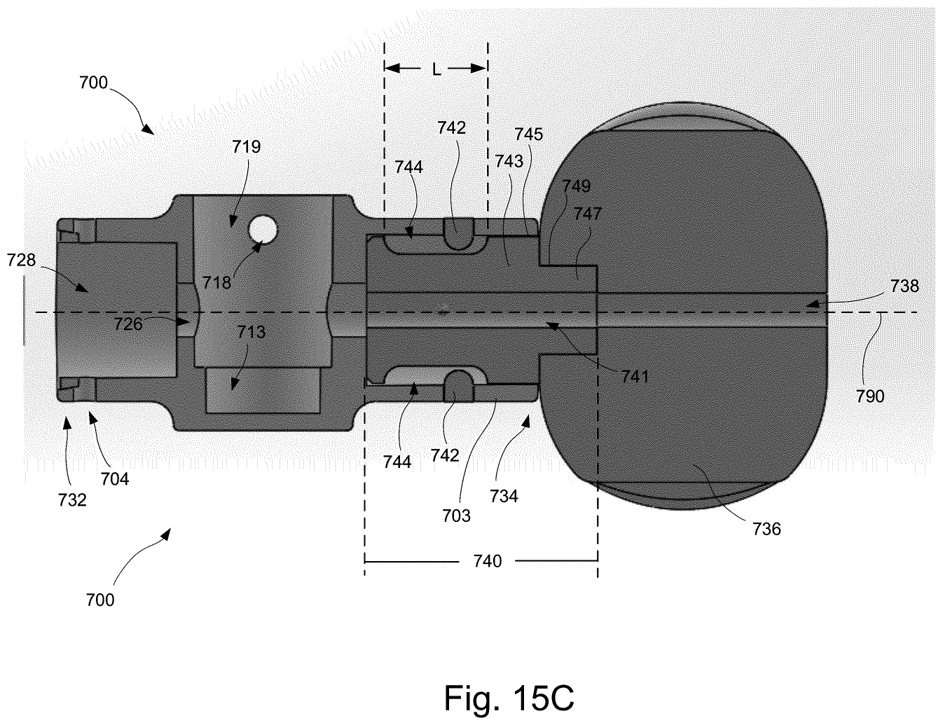

Referring to FIGS. 15A-15C, a securement device 700 provided with a grippable handle 736 is illustrated in accordance with another embodiment of the present technology. Securement device 700 may include a housing 701, a grippable handle 736, and a coupling mechanism 740. The housing 701 may include a distal end 732 and a proximal end 734. The distal end 732 may be configured to couple with a fixation device, such as fixation device 130. In some aspects, distal end 732 couples with fixation device 130 via a hemostasis valve 131. As will be described in greater detail, the coupling mechanism 740 rotatably couples the distal end of grippable handle 736 to the proximal end 734 of housing 701 such that handle 736 is rotatable relative to housing 701. The housing 701, coupling mechanism 740, and handle 736 each extend along a longitudinal axis 790 (shown in FIG. 15C). The longitudinal axis 790 extends proximally and distally.

Securement device 700 may include any of the features in relation to securement devices described herein. Thus, housing 701 is configured to be used with the button 2302, spring, flexible sleeve, and retention pin 2306 to selectively restrict or permit movement of the catheter 102 through the housing 701 in the same manner described herein in relation to securement devices.

In one aspect, the coupling mechanism 740 includes a substantially cylindrical component 743 and one or more pins 742. The proximal end 734 of the housing 701 includes a collar 703 including a cylindrical cavity 745 configured to receive at least a portion of cylindrical component 743. The cylindrical component 743 is translatable along the longitudinal axis 790 relative to housing 701 and rotatable about axis 790 relative to housing 701 within the cavity 745. The cylindrical component 743 includes a slot 744 formed on the outer cylindrical circumference of the cylindrical component 743. The pins 742 extend from an interior surface of collar 703 into the slot 744.

The proximal end of cylindrical component 743 is attached to the distal end of handle 736 such that when a user pushes or pulls handle 736 to translate handle 736 along axis 790, the cylindrical component 743 is also translated along axis 790 into or out of the cavity 745 in collar 703. Moreover, component 743 and handle 736 are attached such that when a user rotates handle 736 (clockwise or counterclockwise) about axis 790, the cylindrical component 743 also rotates about axis 790. In one aspect, the cylindrical component 743 may include a proximal projection 747 and the distal end of the handle 736 may include a cavity 749 configured to receive the projection 747. The projection 747 may be inserted into the cavity 749 and attached to the surfaces forming cavity 749 to attach component 743 to handle 736.

As shown in FIG. 15C, the slot 744 has a length L from a distal end of the slot 744 to a proximal end of the slot 744. As handle 736 is translated along axis 790, the pins 742 abut against the distal and proximal ends of the slot 744 to define the length L of translation that the coupling mechanism 740 permits the handle 736 and cylindrical component 743 to travel along axis 790. In one aspect, the length L is in a range of 0.5 to 4 centimeters (cm). In another aspect, the length L is in a range of 1 to 2 cm. It will be appreciated that other suitable lengths may be chosen. In one aspect, as shown in FIG. 15C, when the handle 736 is fully translated in the distal direction along axis 790, the distal end of the cylindrical component abuts the housing 701 at the distal end of cavity 745. As will be appreciated, in such embodiments, the user may translate the position of the pump (e.g., back and forth) at the same time or at different times to facilitate rotation and proper positioning of the pump.

In one aspect, the slot 744 extends around the entire circumference of cylindrical component 743. In this aspect, coupling mechanism 740 permits handle 736 to be rotated about axis 790 (in either direction) by any amount desired by the user (e.g., 90 degrees, 180 degrees, 360 degrees, 720 degrees etc.) In another aspect, slot 744 may be configured to limit the amount that handle 736 may be rotated to limit the amount of torque that may be placed on catheter 102. For example, the slot 744 may be configured to only extend around the circumference of cylindrical component 743 by a predetermined number of degrees rotation (e.g., 90 degrees, 120 degrees, 180 degrees, etc.) such that the pin 742 will abut against the sides of the slot to restrict rotation of handle 736 within defined rotational limits. Alternatively, slot 744 may include one or more dividers (not shown) extending longitudinally in slot 744. The pins 742 may abut against these dividers to restrict the rotation of handle 736 within defined rotational limits.

The handle 736 may include an interior lumen 738, which extends along the longitudinal axis 790 and is configured to receive a portion of catheter 102. As described herein, the lumen 738 may have a diameter that is larger than outer diameter of catheter 102. The cylindrical component 743 includes an interior lumen 741, which extends along the longitudinal axis 790 and is configured to receive at least a portion of catheter 102. In one aspect, when handle 736 is not compressed and not deformed, the lumen 738 may have a diameter that is the same as the diameter of lumen 741. Lumens 738 and 741 may be disposed adjacent to each other to form a longer lumen extending from the proximal end of handle 736 to the distal end of component 743.

As described in relation to handles 536 and 636, handle 736 also may be deformable in response to a compression force applied to the handle 736 to thereby deform lumen 738. The compression force may be applied by a user gripping the exterior surface 737 of handle 736 and squeezing the handle 736. Alternatively, as described herein, handle 736 may include an actuator that may be actuated by the user to apply the compression force to handle 736.

The catheter 102 may be inserted through lumens 738 and 741 and through the securing mechanism 719 that may include securing structures described elsewhere herein (e.g., button 2302, a sleeve, etc.) of device 700. Then, a user may compress handle 736 to deform lumen 738 such that the interior surface of handle 736 that forms lumen 738 grips the exterior of the portion of catheter 102 disposed in lumen 738. With the handle 736 gripping the catheter 102 and the securing mechanism of device 700 not restricting the movement of catheter 102, the user may torque handle 736 to rotate handle 736. The torque applied to handle 736 may then be transferred to catheter 102 to rotate catheter 102. Moreover, with the handle 736 gripping the catheter 102 and the securing mechanism 719 of device 700 not restricting the movement of catheter 102, the user may translate the handle 736 along the longitudinal axis 790 by pushing or pulling handle 736 proximally or distally. The translation of handle 736 is transferred to catheter 102 to push or pull catheter 102 along axis 790. It is to be appreciated that securing device 700 is configured such that the user is permitted to rotate and longitudinally translate catheter 102 simultaneously (or at separate times) using handle 736. In this way, handle 736 may be used to torque, push, and/or pull the catheter 102 to steer pump 101. The handle 736 may be made of a silicone, rubber, or other suitable material that is deformable and configured to grip catheter 102 when compressed. The handle 736 may be configured to return to its original state/shape (prior to compression) when the compression force is removed from handle 736.

As shown, the handle 736 may be of a sufficient size and length and may have a shape which aids the user to grip and rotate handle 736. As best seen in FIG. 15A, the exterior surface 737 of handle 736 may include one or more concave and/or convex portions that aid the user to grip and apply torque to handle 736.

It is to be appreciated that, in some aspects, when the securing mechanism of device 700 (e.g., including button 2302) is not engaged to restrict movement of catheter 102 and the handle 736 is not compressed, the securing device 700 and the catheter 102 are displaceable relative to each other on axis 790. In this aspect the diameters of lumens 738, 741 and the lumen of the flexible sleeve are larger than the outer diameter of catheter 102.

As described above in relation to FIG. 13, the securement device 700 may be provided with a second coupling mechanism configured to rotatably couple the proximal end of the grippable handle 736 to the protective sleeve 136.

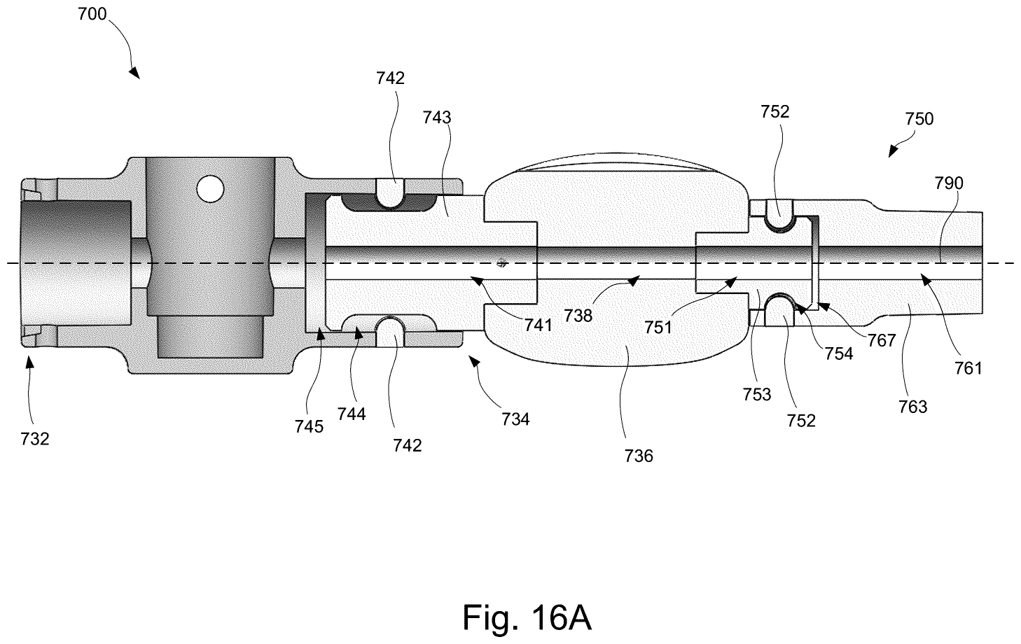

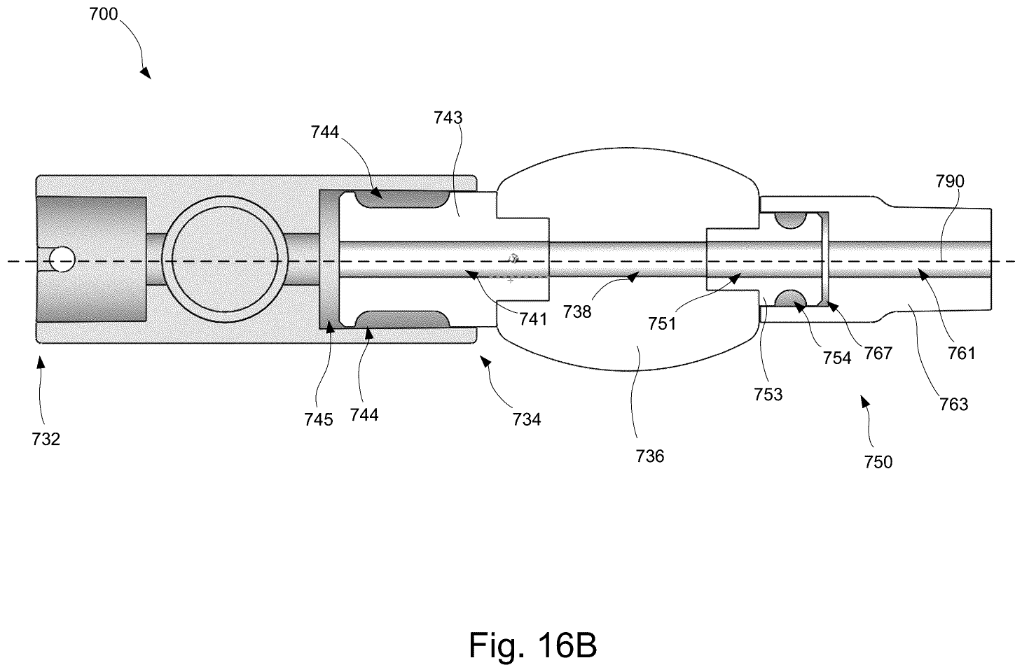

For example, referring to FIGS. 16A-16B, securement device 700 is illustrated with a coupling mechanism 750 for rotatably coupling the proximal end of handle 736 with protective sleeve 136 (not shown) in accordance with the present technology.

In one aspect, the coupling mechanism 750 may include cylindrical components 753, 763, and one or more pins 752. The distal end of component 753 is attached to handle 736 such that, when handle 736 is rotated, component 753 is rotated. The proximal portion of component 753 may include a circumferential slot 754 formed in the outer surface of component 753 and extending around the circumference of component 753.

Component 763 includes an interior cavity 767 at the distal end of component 763 that is configured to receive the proximal portion of component 753. The component 753 is rotatable within the cavity 767 (but such rotation need not translate to component 763) when handle 736 is rotated. Pins 752 extend from an interior surface forming the cavity 767 toward the axis 790 and into the slot 754. The pins 752 and slot 754 are configured to permit components 753, 763 to rotate about axis 790 relative to each other, but to prevent components 753, 763 from being separated longitudinally along axis 790 from each other.

The proximal portion of component 763 may be configured to couple with protective sleeve 136 (not shown). In some aspects, the proximal portion of component 763 has a smaller outer diameter than the distal portion of component 763. The distal end of sleeve 136 may be disposed over the exterior of the proximal portion of component 763 and coupled to component 763. The proximal portion of component 763 and protective sleeve 136 may be coupled or attached using any suitable technique. For example, protective sleeve 136 may be bonded to the proximal portion of component 763 or affixed thereto using adhesives, etc. Protective sleeve 136 may be removably coupled to the proximal portion of component 763 using any suitable mechanical coupling, such as a ring mounted on the proximal portion of component 763.

Component 753 includes an interior lumen 751 extending along the longitudinal axis 790 and component 763 includes an interior lumen 761 extending along the longitudinal axis 790. Lumens 751, 761 are configured to receive a portion of catheter 102 within each lumen. In one aspect, lumens 751, 761 each have the same diameter as lumen 738 (when handle 736 is not compressed) and lumen 741. As shown, lumens 741, 738, 751, 761 are disposed adjacent to each other along axis 790 and together form a larger lumen that receives catheter 102.

The securing device 700 shown in FIGS. 16A and 16B with coupling mechanism 750 may allow the protective sleeve 136 to be coupled to the distal end of the securement device 700 and thus prevent contamination of catheter 102 as it is advanced in the distal direction for insertion into the patient's vasculature. At the same time, coupling mechanism 750 permits handle 736 to be rotated relative to sleeve 136 such that sleeve 136 is not torqued and damaged when handle 736 is used to torque catheter 102.

In other aspects of the present technology, instead of modifying the securement device to aid in the user's ability to grip catheter 102, a gripping device that is separate from the securement device of the assembly may be provided with enhanced gripping features.

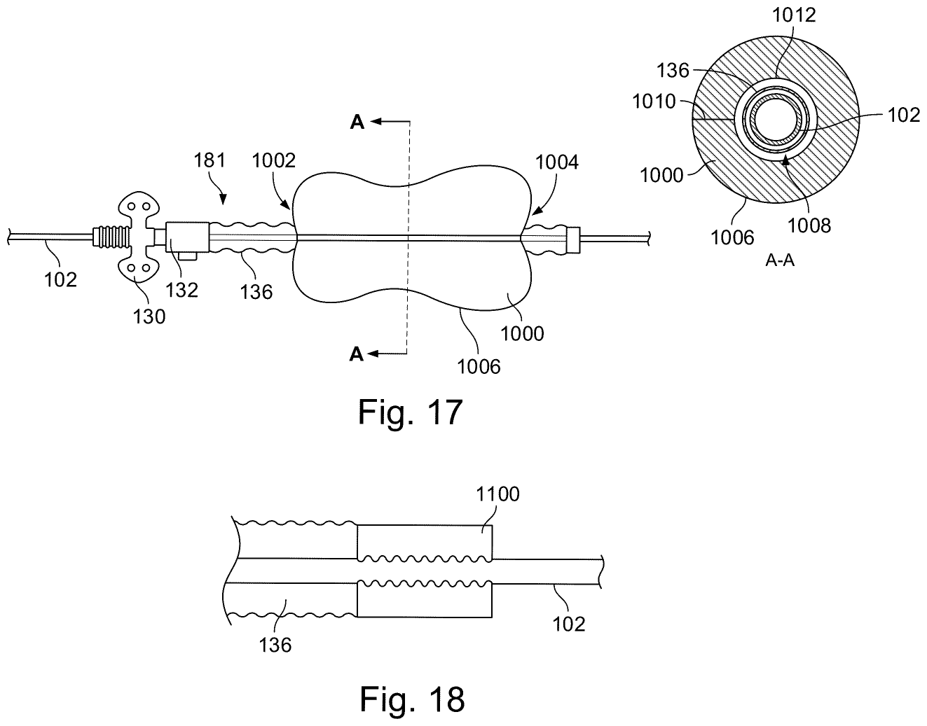

For example, referring to FIG. 17, in one aspect, a gripping device 1000 may be provided that is configured to grasp a portion of catheter 102, e.g., over protective sleeve 136 at portion 181 of catheter 102, exterior to the patient. In the aspect shown in FIG. 17, the gripping device 1000 may be configured as a handle that can be gripped by the user. For example, the gripping device 1000 includes a distal end 1002, a proximal end 1004, an exterior surface 1006, and an interior surface 1012 defining an interior lumen 1008 extending through the interior of gripping device 1000 from the distal end 1002 to the proximal end 1004. The interior lumen 1008 has a diameter selected to permit the interior lumen to receive a portion of catheter 102 and protective sleeve 136. In some aspects, the diameter of lumen 1008 is larger (e.g., in a range of 1% to 20% larger) than the outer diameter of catheter 102. In response to a gripping pressure (i.e., a compression force) applied to the exterior 1006 of gripping device 1000, gripping device 1000 is configured to deform such that the interior surface 1012 grips the exterior of sleeve 136 and catheter 102. The deformation of gripping device 1000 may decrease the diameter of at least a portion of the interior surface 1012 along at least one axis that is perpendicular to the longitudinal axis along which the interior lumen 1008 extends from the proximal end 1004 to the distal end 1002. In this way, at least a portion of the interior surface 1012 grips the exterior surface of catheter 102 such that any torque, pushing, or pulling force applied by a user to gripping device 1000 is transferred to catheter 102. Thus, by gripping and squeezing the gripping device 1000, a user may push, pull, or torque catheter 102 to position the blood pump 101 as desired within the patient. In some aspects, after the user stops gripping and squeezing gripping device 1000, gripping device 1000 is configured to return to its original, unstressed state/shape prior to deformation.

The gripping device 1000 may be made of rubber, silicone, or another material suitable for gripping objects and for being deformable, and in some aspects, reversibly deformable. The gripping device 1000 has a sufficient length from end 1004 to end 1002 to permit a stable grasping of gripping device 1000 and the catheter 102 (and/or sleeve 136) so that the user can push, pull, or torque catheter 102 to steer and position the pump 101 as desired in the patient. In some embodiments, the gripping device may be between 1 and 6 inches, although other suitable lengths may be chosen. In one aspect, handle 1000 may be configured to be removeable from sleeve 136 and catheter 102. In this regard, the gripping device may include an accessory attachable to the catheter. For example, handle 1000 may include a longitudinal slit 1010 (or a longitudinal gap) extending from a proximal to a distal end of handle 1000 that is configured such that the user can part the slit 1010 (or gap) to open the gripping device 1000 such that a portion of catheter 102 and/or sleeve 136 can be received by or removed from lumen 1008. As described above, with the catheter 102 and/or sleeve 136 received into the gripping device 1000, the user may grip the gripping device 1000 to apply sufficient gripping pressure (a compressive force) to the catheter 102 to torque, pull, or push the catheter as desired. In some aspects, the exterior 1006 of the gripping device 1000 may have an ergonomic shape (e.g., comprising concavely and/or convexly shaped portions of the exterior surface) to aid gripping.

Referring to FIG. 18, in another aspect, a gripping device 1100 is provided in accordance with the present technology. Gripping device 1100 may include any of the features described above in relation to gripping device 1000. As shown in FIG. 18, the gripping device 1100 may be disposed over catheter 102 (e.g., at portion 181 exterior to the patient) and configured to provide a stable gripping of the catheter 102 when a user applies gripping pressure (a compressive force) to the exterior of gripping device 1100. As described in relation to gripping device 1000, the gripping device 1100 may also be configured to deform in response to a compressive force applied by the user to the exterior of gripping device 1100 such that at least a portion of the internal lumen of gripping device 1100 deforms and the interior surface forming the lumen grips the exterior of catheter 102. In this way, when gripping the gripping device 1100, the user may push, pull, or torque to the catheter 102 to position the blood pump 101 appropriately within the patient. In one aspect, the gripping device 1100 may be integrated with sleeve 136. For example, in some aspects, the gripping member 1100 may be coupled to, attached, or integrated at an end (proximal or distal) of sleeve 136, as shown. In other aspects, gripping member 1100 may be integrated with sleeve 136 at a position between the ends of sleeve 136. The gripping member 1100 may be made of rubber, silicone, or any other suitable material. In one aspect, as shown in FIG. 18, the interior surface of gripping device 1100 that forms the interior lumen for receiving catheter 102 may be textured or include a pattern of raised portions configured to aid in gripping the exterior of catheter 102 when gripping device 1100 is gripped and deformed by the user.

It is to be appreciated that the interior lumens of gripping devices 1000, 1100 may each have a diameter when in an uncompressed state that is larger than the outer diameter of catheter 102 (and/or than the outer diameter of catheter 102 with sleeve 136 disposed over catheter 102). In this way, when devices 1000, 1100 are uncompressed (not deformed), devices 1000, 1100 and catheter 102 are slidable relative to each other with a portion of catheter 102 through the interior lumen of devices 1000, 1100.

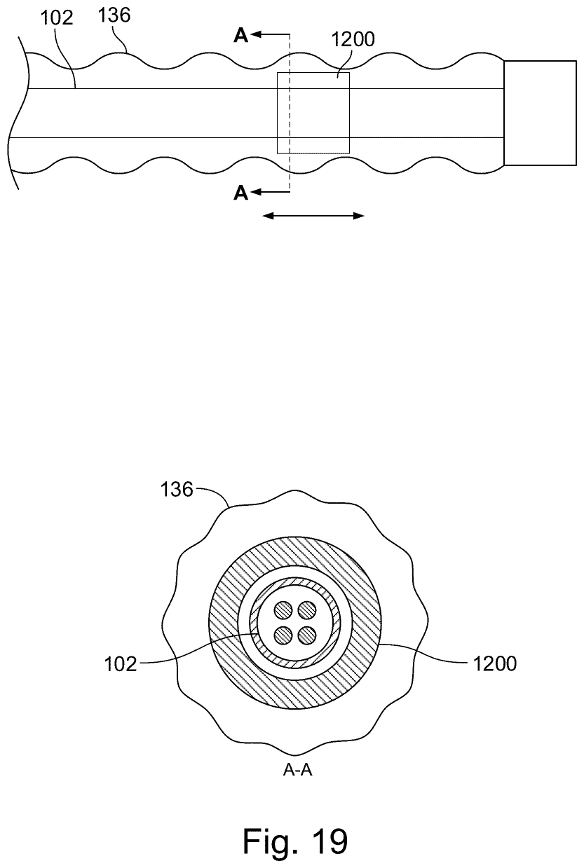

Referring to FIG. 19, in another aspect, a gripping device 1200 is provided in accordance with the present technology. Gripping device 1200 may include any of the features described above in relation to gripping devices 1000, 1100. In the aspect illustrated in FIG. 19, the gripping device 1200 is formed as a ring or tube with an interior lumen extending from the proximal end to the distal end of device 1200. Gripping device 1200 is disposed over the exterior of catheter 102 (e.g., at portion 181 of FIG. 17) and interior of sleeve 136. In one aspect, device 1200 is disposed between catheter 102 and sleeve 136 and the distal and proximal ends of sleeve 136 are coupled or fixed to the assembly 100, as described above. In one aspect, gripping device 1200 is configured to be freely slidable over catheter 102 when the user is not applying gripping or compression force to device 1200. In this regard, the inner diameter gripping member 1200 when uncompressed is larger than the outer diameter of catheter 102. Gripping member 1200 is configured such that, when gripping pressure is applied to gripping member 1200 by a user, the gripping member 1200 grips the exterior of catheter 102 and permits the user to push, pull, or torque the catheter 102 to position the blood pump 101 appropriately within the patient. The gripping member 1200 may be made of rubber, silicone, or any other suitable material.

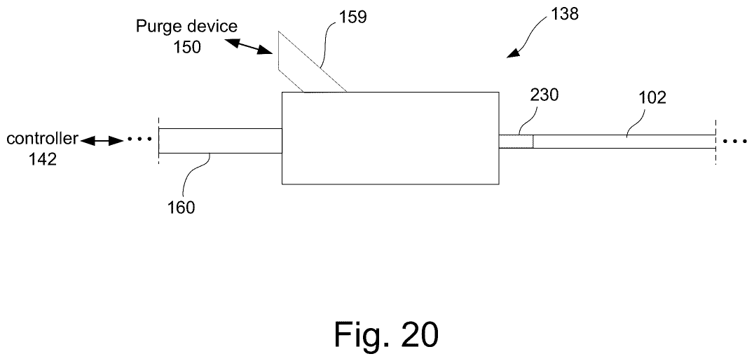

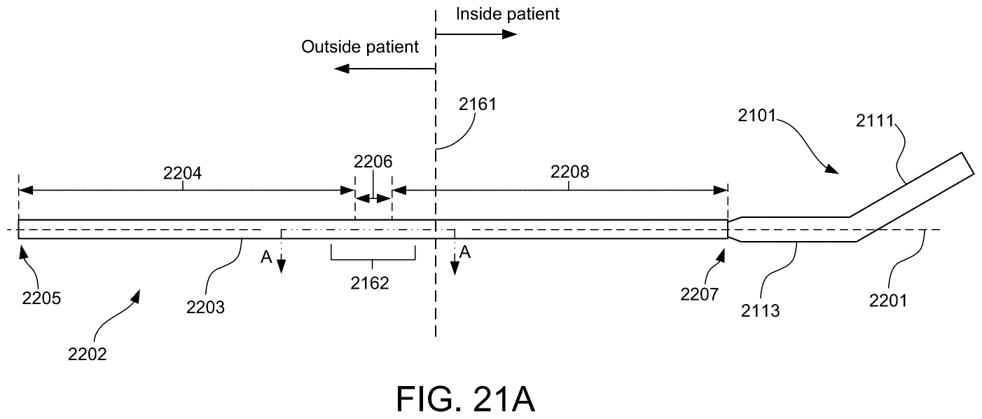

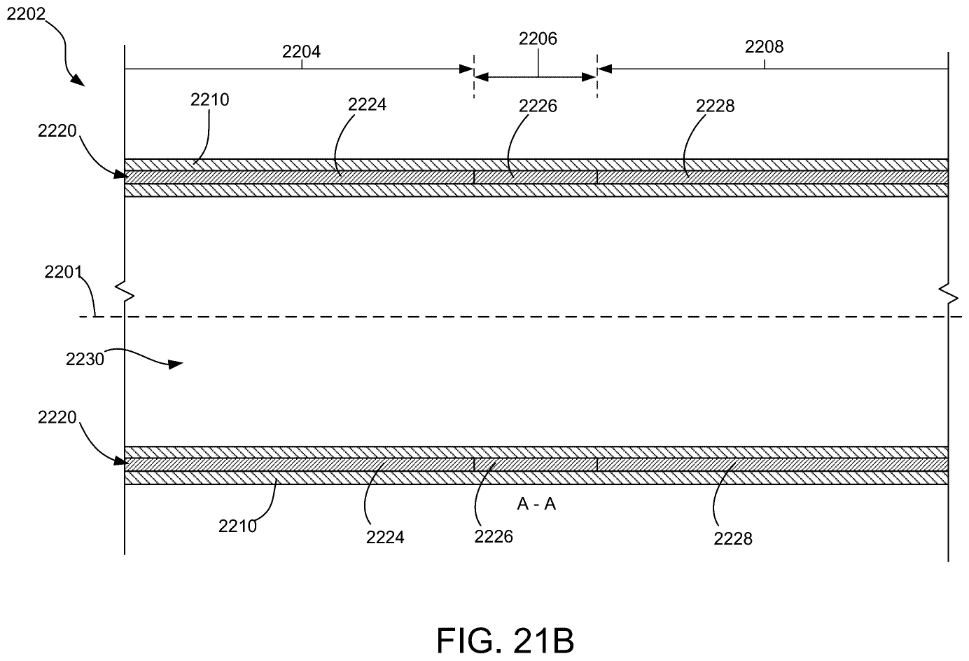

In any of the aspects described herein and shown in FIGS. 11-19 relating to improved gripping of catheter 102 or described below in FIGS. 21A, 21B relating to improving the torque transfer characteristic of catheter 102, the proximal end of the catheter 102 may be coupled to a portion of assembly 100, e.g., such as handle 138, with a coupling mechanism, such as coupling mechanism 230 (configured as described in relation to FIG. 3A or FIG. 3B) described above to permit the distal end of catheter 102 to rotate freely (or within predetermined limits) relative to handle 138. The coupling mechanism may be a rotary joint, such as a slip ring or other rotatory joint that permits wires, fibers, and purge line to pass through or be coupled by the rotary joint. For example, as shown in FIG. 20, coupling mechanism 230 may couple the proximal end of catheter 102 to handle 138 and permit the wires, fibers, and purge line of assembly 100 to pass through (or in the case of the wire by electrically coupled by) coupling mechanism 230 into catheter 102. In this arrangement, when the user grips catheter 102 (e.g., using any of the gripping members or securing devices described herein) and torques catheter 102, the strain at the proximal portion of catheter 102 is reduced and the mass of handle 138 will hinder the angular rotation of catheter 102 less.

It is to be appreciated that, in any of the aspects described herein, the catheter 102, 202 may be modified to enhance its ability to transfer torque and resist kinking and thus aid in steering and positioning blood pump 101, 201.

For example, in one aspect, catheter 102, 202 may include a suitably stiff backbone that increases the ability of catheter 102, 202 to transfer torque applied to catheter, e.g., using any of the steering mechanisms, gripping members, and/or securing devices described herein.

In another aspect, catheter 102, 202 may be modified to integrate predetermined structure(s) in the wall of the catheter 102, 202 to enhance the torque transfer characteristics of the catheter 102, 202.

Described herein are structures and methods that improve the torque transmission characteristics of catheters for catheter assemblies. For example, the structures and methods described herein may improve the torque transmission characteristics of catheters, such as, catheter 102 in order to steer pump 101 within the patient. The present technology provides a catheter for a catheter assembly including a reinforcement structure embedded in a wall or layer of the catheter. The reinforcement structure may be configured such that a distal portion of the catheter transmits torque toward the distal end of the catheter within acceptable levels of efficiency. Moreover, the reinforcement structure may be configured such that a proximal portion of the catheter transmits torque less efficiently (i.e., reduces torque transmission) toward the proximal end of the catheter. In this way, the distal portion of the catheter including the reinforcement structure in accordance with the present technology may improve the user's ability to accurately torque the catheter to thereby torque and steer the pump. Moreover, the proximal portion of the catheter including the reinforcement structure in accordance with the present technology may reduce resistance to torquing the catheter.

For example, referring to FIGS. 21A and 21B, a catheter 2202 is shown coupled to blood pump 101 for use with assembly 100 in accordance with aspects of the present technology. As will be described in greater detail, catheter 2202 may include a reinforcement structure 2220 (see FIG. 21B) in accordance with the present technology for improving the torque transmission characteristics of catheter 2202. It is to be appreciated that catheter 2202 may be used with a catheter assembly, such as an intracardiac catheter assembly, or other types of catheter assemblies inserted into a patient. Moreover, it is to be appreciated that catheter 2202 is not shown to scale in FIGS. 21A and 21B.

As shown, catheter 2202 comprises an elongate tubular body 2203 extending along a longitudinal axis 2201 from a proximal end 2205 to a distal end 2207 of body 2203. In one aspect, proximal end 2205 is coupled to plug or handle 138 and distal end 2207 is coupled to the proximal end (e.g., to proximal portion 113) of blood pump 101. The tubular body 2203 includes at least one lumen 2230 extending from proximal end 2205 to distal end 2207 along axis 2201. It is to be appreciated that, in some aspects, catheter 2202 may include a plurality of lumens extending between proximal end 2205 and distal end 2207 to carry electrical wires, purge lines, optical fibers, and/or other wires/lines of assembly 100.

Body 2203 comprises a layer 2210 forming the jacket of body 2203. In one aspect, layer 2210 forms the inner and outer surfaces of body 2203. The layer 2210 may be made of a polymer, such as polyurethane. Body 2203 further comprises a reinforcement structure 2220 embedded in layer 2210. In one aspect, layer 2210 may encapsulate reinforcement structure 2220. Reinforcement structure 2220 extends within layer 2210 along longitudinal axis 2201 from the proximal end 2205 to the distal end 2207 of body 2203. In one aspect, reinforcement structure 2220 may have an elongate tubular shape and is coaxially arranged with layer 2210 along axis 2201. Reinforcement structure 2220 may be made of a metallic material. In some aspects, reinforcement structure 2220 is made of a metal alloy, such as nitinol. Body 2203 with reinforcement structure 2220 may be configured to be sufficiently flexible to permit catheter 2202 to bend within the vasculature of a patient.

Body 2203 may comprise a proximal portion 2204 and a distal portion 2208. As will be described in greater detail, the reinforcement structure 2220 may be configured such that the proximal portion 2204 of body 2203 has different torque transmission characteristics than the distal portion 2208 of body 2203.

As shown in FIG. 21B, the reinforcement structure 2220 may include a proximal portion 2224 and a distal portion 2228. The proximal portion 2224 extends within layer 2210 along axis 2201 from the proximal end to the distal end of proximal portion 2204 of body 2203. The distal portion 2228 may extend within layer 2210 along axis 2201 from the proximal end to the distal end (i.e., distal end 2207) of distal portion 2208 of body 2203.

Proximal portion 2224 of reinforcement structure 2220 may be configured to reduce the amount of torque transmitted from one end of proximal portion 2204 of body 2203 to an opposite end of proximal portion 2204. In one aspect, proximal portion 2224 is configured as a spiral or coil (e.g., a nitinol coil) that is wound around axis 2201. The coil portion 2224 of reinforcement structure 2220 may provide kink resistance to portion 2204 of body 2203, while at the same time maintaining flexibility. Furthermore, the coil portion 2224 may be configured such that, proximal portion 2204 transmits torque inefficiently over the length of portion 2204. In one aspect, proximal portion 2204 is configured to transmit 1-20% of the torque inputted at the distal end of proximal portion 2204 to the proximal end of portion 2204. Thus, a large rotational displacement at the distal end of portion 2204 (e.g., due to a user torquing catheter 2202) will produce a much smaller rotational displacement at the proximal end of portion 2204 that is coupled to plug or handle 138. Accordingly, the inefficient torque transmission of portion 2204 of body 2203 reduces the amount of resistance a user may experience when applying torque to catheter 2202.

Distal portion 2228 of reinforcement structure 2220 may be configured such that distal portion 2208 of body 2203 transmits a large proportion (e.g., 50%-100%) of torque inputted at one end of distal portion 2208 of body 2203 to an opposite end of distal portion 2208. Thus, distal portion 2208 may be configured to transfer a large proportion (e.g., 50%-100%) of the rotational displacement at the proximal end of portion 2208 (e.g., due to a user torquing catheter 2202) to the distal end 2207 of portion 2208. Accordingly, portion 2208 of body 2203 may reduce the chances of catheter 2202 recoiling after being torqued by a user. Moreover, portion 2208 may permit more accurate torquing of pump 101.

In one aspect, distal portion 228 of reinforcement structure 2220 may be formed by laser cutting the material of structure 2220 (e.g., nitinol) according to a predetermined pattern. The predetermined pattern is selected to impart the desired torque transmission characteristics to distal portion 2208. For example, the predetermined pattern is selected such that distal portion 2208 transmits torque more efficiently than portion 2204 of body 2203. Moreover, the predetermined pattern is selected such that distal portion 2208 is sufficiently flexible to permit insertion and navigation of catheter 2202 into the vasculature of the patient.

In one aspect, the predetermined laser cut pattern comprises a lattice structure.

In one aspect, the predetermined laser cut pattern comprises a plurality of apertures or windows in portion 2228 of reinforcement structure 2220. The apertures may be arranged to form superimposed torque paths along the material of structure 2220 between the apertures. In one aspect, a first torque path and a second torque path may be formed by the material of the reinforcement structure 2220 that is between the apertures. The first torque path and the second torque path may each be spirals that are wound around axis 2201. The first spiral torque path may be wound around axis 2201 in an opposite direction to the second spiral torque path. The first and second torque paths may enable portion 2208 of body 2203 to efficiently transmit torque both when catheter 2202 is torqued clockwise about axis 2201 and when catheter 2202 is torqued counterclockwise about axis 2201. Thus, the predetermined laser cut pattern facilitates bi-directional torque transmission by having a continuous pathway for torsional load to be delivered in opposing directions.

In one aspect, the predetermined laser cut pattern of portion 2228 may include elements or features configured to enhance the flexibility of portion 2208 of body 2203 and minimize length changes under torsional load.

The inventors have recognized that the structure of portion 2228 resulting from the predetermined laser cut pattern may provide several advantages and benefits when implemented in a catheter as described herein. For example, portion 2228 of reinforcement structure 2220 of the present technology can achieve efficient bi-directional torque transmission (i.e., when rotated in either direction) in a single layer by providing the above-described superimposed torque paths. Thus, the overall wall thickness of catheter 2202 may be decreased relative to reinforcement structures including multiple concentric layers.

In one aspect, body 2203 further includes an intermediate portion 2206 disposed between proximal portion 2204 and distal portion 2208. Moreover, reinforcement structure 2220 may further include an intermediate portion 2226 disposed between proximal portion 2224 and distal portion 2228. Intermediate portion 2226 extends within layer 2210 along longitudinal axis 2201 from a proximal end of intermediate portion 2206 of body 2203 to a distal end of intermediate portion 2206 of body 2203. Intermediate portion 2226 may be configured to couple proximal portion 2224 to distal portion 2228. Moreover, intermediate portion 2226 may comprise geometric features configured to promote mechanical connections between intermediate portion 2226 and the layer 2210. For example, the geometric features may comprise protrusions extending from an outer surface of portion 2226 toward the exterior of body 2203. The geometric features may additionally or alternatively comprise apertures in the material (e.g., nitinol) of which the reinforcement structure 2220 is made. In one aspect, the material of layer 2210 (e.g., a polymer) may be reflowed onto reinforcement structure 2220 to form catheter 22202. In this aspect, the geometric features of intermediate portion 2226 promote mechanical interlocking between the geometric features of portion 2226 and the material of layer 2210. In this way, intermediate portion 2226 promotes a secure mechanical connection between reinforcement structure 2220 and layer 2210.

It is to be appreciated that, in some aspects, reinforcement structure 2220 may be bonded chemically to layer 2210. The chemical bond between reinforcement structure 2220 and layer 2210 may be in addition to the mechanical connections (e.g., mechanical interlocking) between reinforcement structure 2220 and layer 2210. Alternatively, the chemical bond between reinforcement structure 2220 and layer 2210 may replace the mechanical connections.

Portions 2204, 2206, and 2208 of body 2203 may each have predetermined longitudinal lengths selected to appropriately position portions 2204, 2206, and 2208 in relation to the patient to take advantage of the different torque transmission characteristics of portion 2204 and 2208 of body 2203. For example, the patient's skin at the access site for the pump assembly is indicated in FIG. 21A by dotted line 2161, which forms a boundary between the exterior of the patient and the interior of the patient. As shown, proximal portion 2204 of body 2203 and proximal portion 2224 of reinforcement structure 2220 may be disposed exterior to the patient when the distal portion of catheter 2202 and the pump 101 are inserted into the patient. Intermediate portion 2206 of body 2203 and intermediate portion 2226 of reinforcement structure 2220 are also disposed exterior to the patient when the distal portion of the catheter 2202 and pump 2101 are inserted into the patient. Described another way, distal portion 2208 of body 2203 and the distal portion 2228 of reinforcement structure 2220 may be predominantly disposed within the patient. As shown, in one aspect, a small length (e.g., 1% to 10% of the total length) of distal portion 2208 of body 2203 and distal portion 2228 of reinforcement structure 2220 is disposed exterior to the patient, with the remainder being introduced into the vasculature of the patient.

As described above, after a catheter of a pump assembly, such as catheter 22202, is inserted into a patient, a user may grip the portion of catheter 22202 that is exterior to the patient and apply torque to catheter 22202 to steer and position pump 101 to a desired position and orientation within the patient. Typically, a user will grip a portion of the catheter 22202 that is exterior to the patient and near the access site (i.e., near dotted line 2161 in FIG. 21A). Where a securement device, such as securement device 132 is used, a user will grip the catheter 22202 near the proximal end of securement device 132. Thus, the user may grip catheter 22202 over intermediate portion 2206 of body 2203 or at least near intermediate portion 2206, such as at the distal end of portion 2204 or at the proximal end of portion 2208. It is to be appreciated that the user's grip may span one or more of the intermediate portion 2206, the distal end of portion 2204, and/or the proximal end of portion 2208. For example, in FIG. 21A, the area 2162 of body 2203 is indicated over which a user may grip and apply torque to catheter 22202.

When a user applies torque to catheter 22202 over area 2162, proximal portion 2204 and distal portion 2208 of body 2203 will each transmit the torque away from area 2162 with differing levels of efficiency. For example, due to the torque transmission characteristics of proximal portion 2224 of reinforcement structure 2220, proximal portion 2204 is configured to transmit a first proportion or ratio of the externally applied torque from the distal end to the proximal end of portion 2204. Moreover, due to the torque transmission characteristics of distal portion 2228 of reinforcement structure 2220, distal portion 2208 may be configured to transmit a second proportion or ratio of the externally applied torque from the proximal end to the distal end of portion 2208 (i.e., distal end 2207). The second proportion of torque transmitted by portion 2208 may be larger than the first proportion of torque transmitted by portion 2204. In one aspect, distal portion 2208 is configured to transmit 50 to 100% of the torque inputted at the proximal end of portion 2208 to the distal end (i.e., end 2207) of portion 2208. In one aspect, proximal portion 2204 may be configured to transmit 1% to 20% of the torque inputted at the distal end of proximal portion 2204 to the proximal end of portion 2204, although the proximal portion may be configured to transmit other suitable percentages or torques.

In one aspect, portions 2224, 2226, and 2228 of reinforcement structure 2220 are interconnected to each other and formed during manufacturing starting from a single tube made of a suitable material (e.g., nitinol) for the reinforcement structure.

It is to be appreciated that in other aspects of the present technology, one or both of portions 2224 and 2228 of reinforcement structure 2220 may have a different structure or configuration than described above.

For example, in one aspect, the proximal portion 2224 may be configured as described above as a spiral or coil (e.g., a nitinol coil) that is wound around axis 2201 in a first direction. However, in this aspect, in contrast to the aspects described above, distal portion 2228 may also be configured as a spiral or coil structure (e.g., made of nitinol) that is configured to transfer torque more efficiently than proximal portion 2224. For instance, in one example of this aspect, the distal portion 2228 may be a spiral or coil (e.g., a nitinol coil) that has a larger gauge winding and/or a different pitch relative to the spiral or coil of portion 2224 such that the distal portion 2228 transfers torque more efficiently than the proximal portion 2224. Additionally, or alternatively, the distal portion 2228 may be configured as a multi-layered concentric coil structure, similar to a torque cable. For example, the distal portion 2228 may comprise two, three, or more distinct, concentric layers of spirally wound coils, which are wound in alternating directions in each successive layer. Such a multi-layered coil structure transfers torque more efficiently than the single-layered coil structure used in the proximal portion 2224.

It is to be appreciated that there are many different combinations of structures/configurations that may be used for the proximal portion 2224 and distal portion 2228 of the reinforcement structure 2220 described herein. However, in all aspects of the present technology, the reinforcement structure 2220 is configured such that the proximal portion 2224 has different torque transfer characteristics relative to the distal portion 2228 such that the distal portion 2228 transfers torque more efficiently than the proximal portion 2224 of the reinforcement structure 2220.

It is to be appreciated that the term “efficiency” as used herein in relation to torque transfer or transmission, describes the proportion, ratio, or amount of externally applied torque that is transferred or transmitted by a region, portion, or length of an elongate structure (such as a catheter, a tube, or other elongate structure) from a first location of the section/region (where the torque is externally applied/inputted) to a second location of the section/region (e.g., distal or proximal of the first location). The “efficiency” may be characterized in terms of a comparison (percentage, ratio, difference) of the inputted torque at the first location, which is externally applied by the user, relative to the torque as measured at the second location. Thus, statements herein regarding a section/region of an elongate structure (e.g., a catheter) that transfers or transmits torque more/less efficiently than another section/region of the elongate structure refer to the differences in the proportion, ratio, or amount of torque that each section of the elongate structure is configured to transmit over its length.

It is to be appreciated that the shaft 220 described herein may be modified to include the structure/features of portion 2228 (e.g., laser cut pattern with superimposed torque paths) of structure 2220 and used to transfer torque between the steering mechanisms described herein (e.g., steering mechanism 224) and the blood pump 201.

In any of the aspects described herein, a controller of the blood pump assembly, such as controller 142 may receive different types of data associated with the position of the blood pump 101, 201 within the patient and the controller 142 may be configured to make determinations and perform different steps based on the received data.

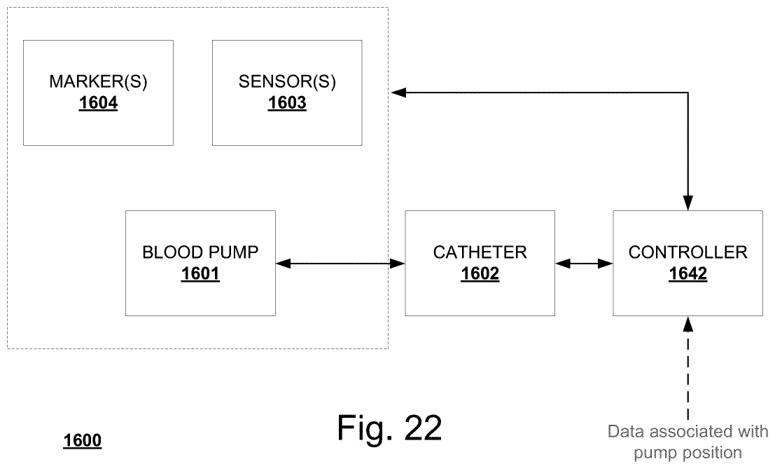

For example, referring to FIG. 22, a blood pump assembly 1600 is shown in accordance with the present technology. The blood pump assembly 1600 may include any of the features and components of the blood pump assemblies 100, 200, steering mechanisms, gripping members, catheters (e.g., including reinforcement structures for enhanced torque transfer) described herein. Blood pump assembly 1600 includes a controller 1642, catheter 1602, blood pump 1601, and one or more position markers 1604 and/or sensors 1603. Sensors 1603 may comprise blood pressure sensors, motor current sensors, and/or one or more position sensors 1603 configured to sense and output data indicative of their position. The position sensors 1603 and/or markers 1604 may be positioned at different locations of blood pump 1601. Although not shown, position markers and sensors may also be disposed at different locations of catheter 1602. Blood pump 1601, catheter 1602, and controller 1642 may include any of the features of the blood pumps, catheters, and controllers described above.

Controller 1642 may be a controller that operates the blood pump 1601, for example, by controlling a motor that rotates an impeller or rotor of blood pump 1601. Although not shown, one or more cables and connectors (such as cable 160 and plug 138, 238) may couple controller 1642 to catheter 1602 and/or blood pump 1601. The catheter 1602 and blood pump 1601 may be inserted into a patient, as described above, such that blood pump 1601 is positioned at an intended location, such as within the patient's heart and surrounding structures (e.g., the aorta and left ventricle).

Controller 1642 may receive one or more different types of data associated with the position of blood pump 1601 within the patient. For example, controller 1642 may receive imaging data (e.g., x-ray images or data derived therefrom) of blood pump 1601. Markers 1604 on blood pump 1601 (and/or catheter 1602) may be identifiable in the imaging data so that the controller 1642 may determine the position of the pump 1601 relative to reference or landmark structures of the patient's body. Moreover, the controller 1642 may receive data from sensors 1603. The sensor data may be pressure data (such as aortic pressure measured by an optical pressure sensor) and motor current, which are indicative of the position of the pump within the patient. The sensor data may further comprise position data from one or more position sensors disposed at different locations on the blood pump 1601. For example, sensors may be disposed distally and proximally on the pump 1601 near the inlet and outlet of the pump 1601. Controller 1642 may receive the data from sensors 1603, markers 1604, etc. via wired or wireless communication(s) with another device (e.g., an image capture device) or directly from the sensors 1603.

Controller 1642 is configured to determine the position of the blood pump 1601 based on the data received associated with the position of the blood pump 1601. The controller 1642 may output the current detected position for display (e.g., on a display such as display 140 or an external display) or output a communication (e.g., a message or notification over a network) including the current detected position to at least one other computing device. Moreover, controller 1642 is configured to determine if the current position of the pump 1601 is acceptable and needs to be adjusted. In this regard, controller 1642 may compare the current position of the pump 1601 to known optimal or desired positions of pump 1601 and/or may recognize known sub-optimal or undesirable (even dangerous) positions that the pump 1601 may be in. For example, an undesirable position for pump 1601 may comprise where the inlet of the pump 1601 is contacting the body tissue within the patient's heart ventricle. If the controller 1642 determines that the pump 1601 is in an unacceptable position within the patient, controller 1642 may notify a user (e.g., by displaying a notification or otherwise) that an adjustment to the position is necessary. The controller 1642 may also determine and notify the user of what type of adjustment to blood pump 1601 needs to be made to orient or position blood pump 1601 as desired. For example, controller 1642 may determine that the distal portion of the blood pump 1601 (including the inlet) needs to be advanced or retracted within the heart or pivoted relative to the proximal portion of the blood pump 1601 (including the outlet). In aspects where assembly 1600 includes a steering mechanism, e.g., such as any of the steering mechanism described herein, the controller 1642 may automatically adjust the position of the blood pump 1601 to a desired position. For example, the controller 1642 may actuate a step motor to torque a drive shaft, such as shaft 220, as described above. Alternatively, the controller 1642 may instruct a user how to manually adjust the position of the pump 1601 using any of the steering mechanisms, gripping members, or catheters described herein.

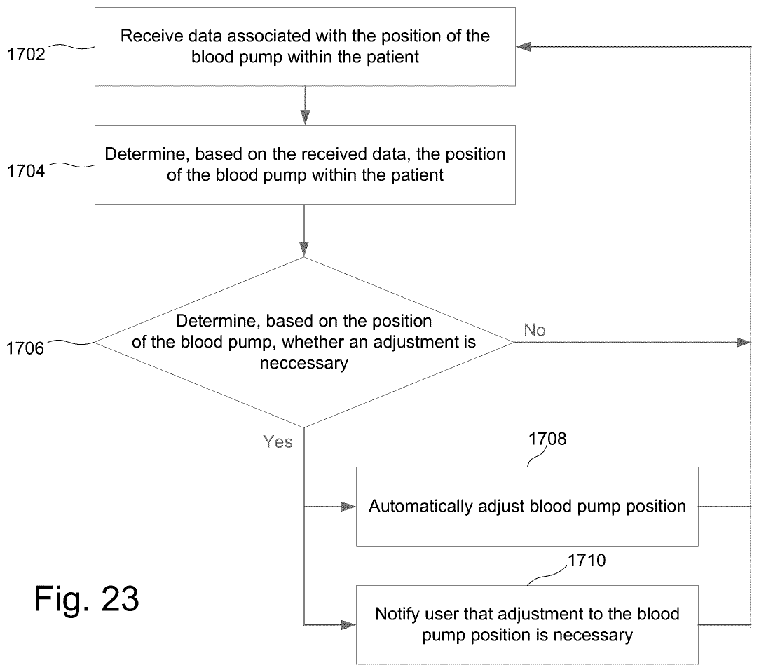

Referring to FIG. 23, a method 1700 is shown in accordance with the present technology. The method 1700 may be performed by assembly 1600.

In step 1702, controller 1642 receives data associated with the position of a blood pump 1601 that is deployed in a patient for pumping blood. As described above, the data may comprise image data or other data associated with detectable position marking, data from one or more sensors, including position sensors on the blood pump 1601, or other data associated with or indicative of the position of the pump 1601 within the patient. In step 1704, controller 1642 determines based on some or all of the received data, the position of the blood pump 1601 within the patient relative to other reference or landmark body structures within the patient. In some aspects, the controller 1642 may display (e.g., on a display like display 140) the determined position for assessment by a user. In step 1706, controller 1642 determines, based on the currently determined position of the blood pump 1601, whether an adjustment to the position of the blood pump 1601 is necessary. For example, if the controller 1642 determines that blood pump 1601 is in a sub-optimal or even dangerous position, controller 1642 may determine an adjustment is necessary. Controller 1642 may further determine, based on an optimal or desired position, what adjustment is necessary to the position of blood pump 1601 to cause blood pump 1601 to change from its current position to the optimal or desired position. If the controller 1642 determines that an adjustment is not necessary (i.e., when the blood pump 1601 has acceptable positioning within the patient), the method returns to step 1702, where the controller 1642 again receives data associated with the position of the blood pump 1601. Alternatively, if the controller 1642 determines that an adjustment to the position of the pump 1601 is necessary, the controller performs one or both of steps 1708 and 1710. If the assembly 1600 has automated steering mechanisms (e.g., like any of the steering mechanisms described herein that may be actuated by a motor, such as a step motor controllable by controller 1642), the controller 1642 may automatically adjust the position of the blood pump 1601 to a more desired position within the patient, in step 1708. In step 1710, controller 1642 notifies the user (e.g., by displaying information on a display, sending one or more notifications to one or more external devices, generating a sound, etc.) that an adjustment to the blood pump 1601 is necessary. In some aspect, the controller 1642 may also communicate or indicate in step 1710 what type of adjustment to the position is necessary.

From the foregoing and with reference to the various figure drawings, those skilled in the art will appreciate that certain modifications may also be made to the present disclosure without departing from the scope of the same. While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

1. An intracardiac blood pump system comprising:
a blood pump comprising a proximal portion and a distal portion;
a catheter coupled to the proximal portion of the blood pump, the catheter having a proximal end and a distal end and an inner lumen extending from the proximal end to the distal end;
a rotatable shaft including a proximal end and a distal end, wherein the shaft extends through the inner lumen of the catheter and the distal end of the shaft is coupled to the proximal portion of the blood pump; and
a steering mechanism coupled to a portion of the shaft, wherein the steering mechanism is configured to rotate the shaft to rotate the blood pump.

2. The intracardiac blood pump system of claim 1, wherein the steering mechanism is proximal of the catheter.

3. The intracardiac blood pump system of claim 1, wherein the steering mechanism comprises a motor configured to rotate the shaft.

4. The intracardiac blood pump system of claim 1, wherein the steering mechanism comprises:
a torque input mechanism; and
a torque transfer mechanism configured to transfer torque inputted to the torque input mechanism to the shaft to rotate the shaft.

5. The intracardiac blood pump system of claim 4, wherein the torque input mechanism comprises a component that is manually rotatable by a user.

6. The intracardiac blood pump system of claim 1, wherein the steering mechanism comprises:
a housing;
a first gear rotatably mounted in the housing and configured to be attached to the portion of shaft;
a gear shaft at least partially disposed in the housing; and
a second gear attached to the gear shaft and disposed within the housing such that the second gear engages the first gear,
wherein when the gear shaft is rotated, the second gear rotates the first gear such that the shaft is rotated.

7. The intracardiac blood pump system of claim 1, wherein the shaft comprises a torque coil.

8. The intracardiac blood pump system of claim 1, wherein the shaft comprises a lumen extending from the proximal end to the distal end of the shaft.

9. The intracardiac blood pump system of claim 8, further comprising at least one of:
one or more conductors extending through the lumen of the shaft and electrically coupled to one or more components of the blood pump; and/or
an optical fiber extending through the lumen of the shaft and coupled to an optical sensor of the blood pump.

10. The intracardiac blood pump system of claim 1, further comprising a coupling mechanism configured to rotatably couple the proximal portion of the blood pump to the distal end of the catheter.

11. The intracardiac blood pump system of claim 10, wherein the coupling mechanism is a slip ring.

12. The intracardiac blood pump system of claim 11, wherein the slip ring electrically couples a first electrically conducting wire to a second electrically conducting wire, the first electrically conducting wire disposed in the inner lumen of the catheter and extending proximally from the slip ring toward the proximal end of the catheter and the second electrically conducting wire extending distally from the slip ring toward the blood pump.

13. The intracardiac blood pump system of claim 12, further comprising a controller, wherein the first electrically conducting wire is electrically coupled to the controller.

14. The intracardiac blood pump system of claim 12, wherein the blood pump further comprises a motor and the second electrically conducting wire is electrically coupled to the motor.

15. The intracardiac blood pump system of claim 12, wherein the blood pump further comprises a sensor and the second electrically conducting wire is electrically coupled to the sensor.

16. The intracardiac blood pump system of claim 1, wherein the blood pump comprises a bend between the proximal portion and the distal portion.

17. An intracardiac blood pump system comprising:
a blood pump comprising a proximal portion and a distal portion;
a catheter coupled to the proximal portion of the blood pump, the catheter having a proximal end and a distal end and an inner lumen extending from the proximal end to the distal end; and
a coupling mechanism configured to rotationally couple the proximal portion of the blood pump to the distal end of the catheter.

18. The intracardiac blood pump system of claim 17, wherein the coupling mechanism comprises a first portion and a second portion, wherein the first portion is configured to be rotatable relative to the second portion, and wherein the first portion is coupled to the proximal portion of the blood pump and the second portion is coupled to the distal end of the catheter.

19. The intracardiac blood pump system of claim 18, wherein the coupling mechanism comprises a bore extending through the coupling mechanism from a proximal end to a distal end of the coupling mechanism.

20. The intracardiac blood pump system of claim 19, further comprising a rotatable shaft including a proximal end and a distal end, wherein the shaft extends through the inner lumen of the catheter and the distal end of the shaft is coupled to the proximal portion of the blood pump.

21. The intracardiac blood pump system of claim 20, wherein the shaft extends through the bore.

22. The intracardiac blood pump system of claim 18, wherein the coupling mechanism is a slip ring.

23. The intracardiac blood pump system of claim 22, further comprising:
at least a first electrically conducting wire coupled to the first portion of the slip ring and extending distally from the first portion toward the blood pump; and
at least a second electrically conducting wire coupled to the second portion of the slip ring and extending proximally from the second portion toward the proximal end of the catheter,
wherein the slip ring is configured to electrically couple the first and second electrically conducting wires.

24. The intracardiac blood pump system of claim 23, further comprising:
a controller, and
wherein the blood pump further comprises a motor, and the first electrically conducting wire is coupled to the motor and the first portion of the slip ring, and the second electrically conducting wire is coupled to the second portion of the slip ring and the controller.

25. The intracardiac blood pump system of claim 23, further comprising:
a controller, and
wherein the blood pump further comprises a sensor and the first electrically conducting wire is coupled to the sensor and the second electrically conducting wire is coupled to the controller.

26. A steering mechanism for steering a blood pump of an intracardiac blood pump system, the steering mechanism comprising:
a housing;
a first gear rotatably mounted in the housing and configured to be attached to a portion of a rotatable shaft of the intracardiac blood pump system;
a gear shaft at least partially disposed in the housing; and
a second gear attached to the gear shaft and disposed within the housing such that the second gear engages the first gear,
wherein when the gear shaft is rotated, the second gear rotates the first gear such that the rotatable shaft of the intracardiac blood pump system is rotated.

27. The steering mechanism of claim 26, wherein a first end of the gear shaft is coupled to a manually rotatable component configured to permit a user to rotate the gear shaft.

28. The steering mechanism of claim 26, wherein a first end of the gear shaft is coupled to a motor configured to rotate the gear shaft.
